(19) United States
(12) Patent Application Publication
(10) Pub. No.: US 2016/0022466 A1
(43) Pub. Date: Jan. 28, 2016

(54) SEQUENTIAL SERIES OF ORTHOPEDIC DEVICES THAT INCLUDE INCREMENTAL CHANGES IN FORM

(71) Applicant: LIM INNOVATIONS, INC., San Francisco, CA (US)

(72) Inventors: Andrew C. PEDTKE, San Francisco, CA (US); Robert Adam GESHLIDER, San Francisco, CA (US); Jesse Robert WILLIAMS, San Francisco, CA (US)

(73) Assignee: LIM INNOVATIONS, INC.

(21) Appl. No.: 14/808,741

(22) Filed: Jul. 24, 2015

Related U.S. Application Data
(60) Provisional application No. 62/028,705, filed on Jul. 24, 2014.

(51) Int. Cl. A61F 5/01; G05B 19/4099

(52) U.S. Cl. CPC A61F 5/0127; G05B 19/4099; G05B 2219/35134

(57) ABSTRACT

A method is described for fabricating a sequential series of orthopedic devices custom designed to change a configuration of a body part of a patient from a pretreatment configuration to a treated configuration. The method involves receiving digital data representing the body part of the patient in the pretreatment configuration. The method continues by generating, using the digital data, a sequential series of digital 3D body part models, including at least an initial body part model representing the pretreatment configuration of the body part, a final body part model representing the treated configuration of the body part, and at least one intermediate body part model representing the body part in an intermediate configuration between the pretreatment and treated configurations. The method further involves fabricating the sequential series of orthopedic devices from the sequential series of digital 3D body part models.

### CROSS-REFERENCE TO RELATED APPLICATIONS

This application claims priority to U.S. Provisional Patent Application No. 62/028,705, entitled “System of orthopedic devices that include incremental changes in form,” as filed on Jul. 24, 2014. That application is hereby incorporated into this present application by this statement of incorporation. Additionally, all other publications, patents and patent applications identified in this specification are herein incorporated by reference to the same extent as if each such individual publication or patent application were specifically and individually indicated to be so incorporated by reference.

### TECHNICAL FIELD

The technology relates to medical devices and methods. More specifically, the technology relates to a series of sequential orthopedic devices and a method for using the series of sequential orthopedic devices to treat a patient in an incremental, sequential manner.

### BACKGROUND

Orthopedic casts, splints, and braces have long been used to help protect and stabilize a broken or fractured bone as it heals, or to aid in the correction of a deformity in a limb or a portion of an axial skeleton. European military surgeons in the 19th century introduced the use of Plaster of Paris in the making of splints and casts, and with various improvements, its use still continues. Plaster casts have been applied to limbs and extremities, as well as to the torso and the lumbar spine, basically to all parts of the body that include bony structure. With the advent of plastics in the mid-20th century, use of polyurethane, thermoplastics, and other polymeric compounds has been introduced. Regardless of the materials used, however, the general practice of creating plaster casts has involved using the patient's injured or deformed body part as a positive mold, casting the compliant material around the positive mold, and allowing it to harden.

In spite of the advent of modern materials and their therapeutic advantages for corrective or supportive healing orthopedic devices such as casts, splints, and bases, all prior art cast systems are based on the use of the affected body part as a positive mold. Further, each of the corrective or supportive healing devices is substantially fixed in form, and a singular one-off device. Typically, in the event of changing anatomy, either by healing, growth, or unexpected eventuality, a new orthopedic device must be created, based on the body part as a positive mold.

In many circumstances, a single, fixed-form cast, brace, or splint is appropriate and sufficient. In other instances, however, such a single fixed form orthopedic device can be limited in terms of its usefulness, particularly when the desired therapeutic result is one that involves a change in the form of a body portion. For example, in some instances, it may not be possible to fix a broken bone into a desired final form in a single orthopedic procedure following a complex break. Another example is that represented by children, whose skeletal structure is growing rapidly. These cases are particularly challenging when casts or braces are used for the correction of a deformity, in which case the corrective treatment period can be of a long-term duration. In such cases, a single, fixed-form cast may be appropriate and therapeutically effective for only a short period of time.

For these challenging orthopedic issues, among others, it may be desirable to have alternatives to a single, fixed-form orthopedic device (as enumerated above) within the array of available orthopedic devices. One of the main drawbacks of a single, fixed-form orthopedic device, as created by a series of individual casts during a course of treatment or healing, is simply the cost of the multiple castings, each casting incurring a separate expense and creating the need for the patient to visit an orthopedic facility each time. Embodiments of the present invention, as disclosed herein, may provide a cost effective therapeutic benefit to patients for whom a single, fixed form orthopedic cast insufficiently addresses their needs.

### SUMMARY

In one aspect, a sequential series of individual ankle foot orthotic (AFO) devices are provided, which are custom fitted for a foot of a patient and vary incrementally from one to the next in a flexion angle. The series of devices includes an initial device, a final device, and one or more intermediate devices. Each individual AFO device of the sequential series includes a posterior strut defining a vertical axis and having a proximal end and a distal end; and a foot support portion coupled with the posterior strut at or near its distal end, where the foot support portion is custom made and fitted for the foot of the patient. The posterior strut is not necessarily custom made, but instead may be drawn from an inventory of components with sufficient diversity that it is custom fitted to the patient. The foot support portion of the AFO device includes a foot bed, including a bottom surface defining a bottom plane of the individual AFO device, and an ankle cover removably connected to the foot bed. In such configuration, a vertex of the vertical axis of the posterior strut and the bottom plane of the foot bed defines the flexion angle. An overall configuration of each individual AFO device of the sequential series of AFO devices is determined at least in part by a single digital profile of the foot of the patient.

In some embodiments, the difference between the flexion angles of the initial device and the final device, respectively, is between 80° and 110°, and an incremental flexion angle difference between individual sequential neighboring AFO devices within the sequential series is between 1° and 10°. In some embodiments, a configuration of a distal portion of the posterior strut varies between at least some of the AFO devices in the sequential series, and the varied configuration of the distal portion affects, contributes to, or entirely accounts for the flexion angles of the AFO devices. Optionally, each individual AFO device may further include a leg support connected to a proximal portion of the posterior strut.

In some embodiments, the posterior strut of each individual AFO device includes a thermoplastic composition. In particular examples of these embodiments, the thermoplastic composition includes a thermoplastic fiber composite composition, the fiber including continuous fiber. In some embodiments, the posterior strut of each individual device is drawn from a collection of posterior struts that vary in size and shape. In some embodiments, the foot support portion of each individual AFO device includes a material selected from the group consisting of a thermoplastic composition and a thermoset composition. In some embodiments, the foot bed portion and/or the ankle cover portion of each individual AFO device includes a thermoplastic composition and is formed by a direct molding process against the foot of the patient.

In some embodiments, the foot bed portion and/or the ankle cover portion of each individual AFO device is formed by way of a 3D printed mold, the 3D printed mold being derived from the single digital profile of the foot. In some embodiments, the foot bed portion and/or the ankle cover portion of each individual AFO device is formed by way of a 3D printing process, as directed by the single digital profile of the foot.

In another aspect, a sequential series of individual AFO devices are custom fitted for a foot of a patient and vary incrementally from one to the next in a flexion angle, the series of devices comprising an initial device, a final device, and one or more intermediate devices. Each individual AFO device of the sequential series includes an integrated posterior support/foot bed portion that defines the flexion angle and an ankle cover portion removably coupled with and disposed over the integrated posterior support/foot bed portion. In these embodiments, the posterior support/foot bed portion and the ankle cover portion are both custom made and, accordingly, custom fitted for the foot of the patient, and a single digital profile of the foot of the patient serves as a model for the fabrication of each individual AFO device within the sequential series of the AFO devices.

In some embodiments, a difference between the flexion angles of the initial device and the final device, respectively, is between 80° and 110°, and wherein an incremental flexion angle difference between individual sequential neighboring AFO devices within the sequential series is between 1° and 10°. In some embodiments, the posterior support/foot bed portion of each individual AFO device includes a thermoplastic composition. In some of these embodiments, the thermoplastic composition includes a thermoplastic fiber composite composition, the fiber including continuous fiber. And in some particular embodiments, substantially all of the fiber of the composition is continuous fiber. In some embodiments, the posterior support/foot bed portion and/or the ankle cover portion of each individual AFO device includes a thermoplastic composition and is formed by a direct molding process against the foot of the patient. In some embodiments, the posterior support/foot bed portion and/or the ankle cover portion of each individual AFO device is formed by way of a 3D printed mold, the 3D printed mold being derived from the single digital profile of the foot. In some embodiments, the posterior support/foot bed portion and/or the ankle cover portion of each individual AFO device is formed by way of a 3D printing process, as directed by the single digital profile of the foot.

In another aspect, a method of fabricating a sequential series of individual orthopedic devices for an individual patient may be provided, where the individual orthopedic devices vary incrementally from one to the next in an aspect of form. Embodiments of this method relate to fabricating a sequential series of orthopedic devices custom designed to change a configuration of a body part of a patient from a pretreatment configuration to a treated configuration. Such method embodiments involve: receiving digital data representing the body part of the patient in the pretreatment configuration; generating (using the digital data) a sequential series of digital 3D body part models, including at least an initial body part model representing the pretreatment configuration of the body part, a final body part model representing the treated configuration of the body part, and at least one intermediate body part model representing the body part in an intermediate configuration between the pretreatment and treated configurations; and fabricating the sequential series of orthopedic devices from the sequential series of digital 3D body part models.

In particular embodiments, the sequential series of orthopedic devices includes a sequential series of AFO devices, where the initial, final and at least one intermediate body part models vary, relative to one another, in a flexion angle. The flexion angle change throughout the sequential series of AFO devices may be between 80° and 110° in some embodiments, and an incremental difference between any two adjacent devices within the sequential series is between 1° and 10°.

In some embodiments, receiving the digital data includes receiving 3D imaging data acquired using an imaging modality selected from the group consisting of CT and MRI. In some of these embodiments, receiving the digital data includes receiving a 3D profile of the body part in the form of an STL file. And in some of these embodiments, after the receiving step the method further includes importing the STL file into a CAD application, wherein the generating step is performed using the CAD application. And in some of these embodiments, after the generating step, the method further includes importing the sequential series of body part models into an STL CAD manipulation application.

In various embodiments, the body part of the patient includes an upper limb or a lower limb, and the method further includes repeating the method steps for a contralateral upper limb or lower limb to provide a second sequential series of orthopedic devices for the contralateral upper limb or lower limb. In such embodiments where the sequential series and the second sequential series of orthopedic devices is directed to AFO devices, the two sequential series are configured for left foot and the right foot of the patient.

In various embodiments, fabricating the sequential series of orthopedic devices includes at least one of 3D printing or 3D machining In some embodiments of the method, fabricating the sequential series of orthopedic devices is directed by way of forming a sequential series of positive molds from the sequential series of digital 3D body part models and forming the sequential series of orthopedic devices from the sequential series of positive molds. In other embodiments, fabricating the sequential series of orthopedic devices includes forming a sequential series of negative molds from the sequential series of digital 3D body part models and forming the sequential series of orthopedic devices from the sequential series of negative molds. In yet other embodiments, however, fabricating the sequential series of orthopedic devices includes forming the sequential series of orthopedic devices directly from the sequential series of digital 3D body part models, without using any molds.

Some embodiments of the method further include receiving additional digital data representing the body part of the patient after treatment of the body part has commenced and repeating the generating and fabricating steps to make at least one additional orthopedic device to further treat the body part. Some embodiments of the method further include receiving a treatment plan from a physician, where the treatment plan includes at least one parameter defining the treated configuration of the body part. In some of these embodiments, the method may further include receiving a follow-up or updated treatment plan from the physician during treatment of the patient, where the follow-up treatment plan includes at least one instruction for altering a planned sequential series of orthopedic devices. And in some embodiments, the follow-up treatment plan includes an instruction for receiving a second set of digital data representing the body part of the patient prior to concluding the treatment as originally planned. By way of an example of such an instruction, the follow-up treatment plan may include receiving a second set of digital data representing the body part of the patient prior to concluding the treatment as originally planned. Finally, in some embodiments of the method, the at least one intermediate body part model includes multiple, sequential, intermediate body part models.

Another aspect is directed to a method of making a sequential series of custom-fitted individual AFO device embodiments for an individual patient, the individual AFO devices varying incrementally from one to the next in a flexion angle of the ankle Embodiments of this method include: acquiring a 3D digital profile of the ankle and foot of the patient in the form of an STL file; importing the STL file into a CAD application; and within the CAD application, creating a sequential series of individual digital 3D AFO models, each model including an incremental change in the flexion angle compared to its neighbor within the series, said increment proceeding from an initial plantar flexion angle toward a dorsiflexion angle; importing each model of the sequential series into an STL CAD manipulation application; and fabricating the series of individual AFO devices, as directed by the series of individual 3D AFO models.

In some embodiments, acquiring a 3D digital profile of the ankle and foot of the patient includes acquiring a 3D digital profile of the left and right ankle and foot of the patient. In some embodiments, making a sequential series of individual AFO devices for an individual patient includes making a sequential series of left-right pairs of AFO devices. In some embodiments, fabricating the one or more associated custom-fitted AFO devices includes a process of any of 3D printing or machining In some embodiments, fabricating the one or more associated custom-fitted AFO devices includes forming one or more molds in accordance with the series of individual 3D AFO models, the method further including forming the AFO devices with the one or more molds. In some embodiments, each of the one or more AFO components includes any of a thermoplastic carbon fiber composition or a thermoset resin.

In some embodiments, acquiring a 3D digital profile of the ankle and foot includes acquiring the 3D digital profile only one time, that time being prior to a patient initiating a therapeutic treatment with the series of sequential series of individual AFO devices. In some embodiments, acquiring a 3D digital profile of the ankle and foot includes acquiring the 3D digital profile prior to a patient initiating a therapeutic treatment with the series of sequential series of individual AFO devices, the method further including acquiring one or more further 3D digital profiles after the patient has initiated therapeutic treatment with the series of sequential series of individual AFO devices.

Another aspect is directed to a method of making a sequential series of individual AFO devices for an individual patient, the individual AFO devices varying incrementally from one to the next in a flexion angle of the ankle, the individual devices including a standard sized posterior strut and a custom foot piece. Embodiments of this method include: acquiring a 3D digital profile of the ankle and foot of the patient in the form of an STL file; importing the STL file into a CAD application; and then within the CAD application, creating a sequential series of individual digital 3D AFO models, each model including a standard sized posterior strut and a custom fit foot piece, each model including an incremental change in the flexion angle compared to its neighbor within the series, said increment proceeding from an initial plantar flexion angle toward a dorsiflexion angle; and importing each model of the sequential series into an STL CAD manipulation application. Embodiments of the method continue as selecting the standard sized posterior strut from an inventory of variously sized posterior struts to fit the 3D profile of the ankle and foot, wherein each posterior strut includes an incremental angular change compared to its nearest neighbor in the series, fabricating a custom foot piece, each foot piece including one or more associated custom-fitted AFO components that correspond to each model of the sequential series; and assembling each the one or more associated custom-fitted AFO components and the standard sized posterior strut together to form a series of custom fitted AFO devices, each device within the series varying (as a whole) incrementally from one to the next with regard to the flexion angle.

In some embodiments, the one or more custom-fitted AFO components of the custom fit foot piece includes a foot bed and an ankle cover. In some embodiments, fabricating the one or more associated custom-fitted AFO components includes a process of either 3D printing or machining In some embodiments, each posterior strut includes any of a thermoplastic carbon fiber composition or a thermoset resin. In some embodiments of the method of making a sequential series of AFO devices, each of the one or more of the custom AFO components includes any of a thermoplastic carbon fiber composition or a thermoset resin.

Another aspect is directed to a method of making a sequential series of individual AFO devices for an individual patient, the individual AFO devices varying incrementally from one to the next in a flexion angle (the flexion angle of the ankle or of structural elements of device embodiments), the individual devices including a standard sized posterior strut and a custom foot piece, the custom foot piece being made by way of a mold, the mold being made by way of 3D printing. Embodiments of this method include: acquiring a 3D digital profile of the ankle and foot of the patient in the form of an STL file; importing the STL file into a CAD application; and within the CAD application, creating a sequential series of individual digital 3D AFO models, each model including a standard sized posterior strut and a custom fit foot piece, each model including an incremental change in the flexion angle compared to its neighbor within the series, said increment proceeding from an initial plantar flexion angle toward a dorsiflexion angle. Embodiments of the method continue with importing each model of the sequential series into an STL CAD manipulation application; selecting the standard sized posterior strut from an inventory of variously sized posterior struts to fit the 3D profile of the ankle and foot, wherein each posterior strut includes an incremental angular change compared to its nearest neighbor in the series, fabricating one or more molds by way of 3D printing for one or more associated custom-fitted AFO components that correspond to each model of the sequential series; using the one or more molds, fabricating the associated custom-fitted AFO components; and assembling each the one or more associated custom-fitted AFO components and the standard sized posterior strut together to form a series of custom fitted AFO devices, each device within the series varying (as a whole) incrementally from one to the next with regard to the flexion angle.

In some embodiments, the one or more custom-fitted AFO components include a foot bed and an ankle cover. In some embodiments, fabricating the one or more associated custom-fitted AFO molds includes a process of any of 3D printing or machining In some embodiments of the method of making a sequential series of AFO devices, each strut includes any of a thermoplastic carbon fiber composition or a thermoset resin. And in some embodiments of the method of making a sequential series of AFO devices, each of the one or more AFO components includes any of a thermoplastic carbon fiber composition or a thermoset resin.

Another aspect is directed to method of making a sequential series of individual AFO devices that vary incrementally from one to the next in a flexion angle, the individual AFO devices including a custom foot piece and a custom ankle cover. Embodiments of this method include: acquiring a 3D digital profile of the ankle and foot of the patient in the form of an STL file; importing the STL file into a CAD application; and within the CAD application, creating a sequential series of individual digital 3D AFO models, each model including one or more custom-fitted AFO components, each 3D AFO model including an incremental change in the flexion angle compared to its neighbor within the series, said increment proceeding from an initial plantar flexion angle toward a dorsiflexion angle.

Embodiments of the method continue with importing each model of the sequential series into an STL CAD manipulation application; fabricating one or custom-fitted AFO components by way of 3D printing for one or more associated custom-fitted AFO components that correspond to each model of the sequential series; and assembling each the one or more associated custom-fitted AFO components together to form a series of custom fitted AFO devices, each device within the series varying (as a whole) incrementally from one to the next with regard to the flexion angle. In some embodiments, the one or more custom-fitted AFO components includes a foot bed and an ankle cover. In some embodiments, fabricating the one or more associated custom-fitted AFO components includes a process of any of 3D printing or machining In some embodiments of the method of making a sequential series of AFO devices, each posterior strut includes any of a thermoplastic carbon fiber composition or a thermoset resin. And in some embodiments of the method of making a sequential series of AFO devices, each of the one or more custom-fitted AFO components includes any of a thermoplastic carbon fiber composition or a thermoset resin.

Another aspect is directed to a method of making a sequential series of individual AFO devices for an individual patient, the individual AFO devices varying incrementally from one to the next in a flexion angle (corresponding the flexion angle of a patient's ankle), the individual AFO devices including a custom foot piece and a custom ankle cover, each custom component being formed by way of molds, the molds being formed by way of 3D printing. Embodiments of this method include acquiring a 3D digital profile of the ankle and foot of the patient in the form of an STL file; importing the STL file into a CAD application; and within the CAD application, creating a sequential series of individual digital 3D AFO models, each model including one or more custom-fitted AFO components, each 3D AFO model including an incremental change in the flexion angle compared to its neighbor within the series, said increment proceeding from an initial plantar flexion angle toward a dorsiflexion angle. Embodiments of the method continue with importing each model of the sequential series into an STL CAD manipulation application; fabricating molds for the one or custom-fitted AFO components by way of 3D printing for one or more associated custom-fitted AFO components that correspond to each model of the sequential series; molding custom-fitted AFO components using the fabricated molds; and assembling each the one or more associated custom-fitted AFO components together to form a series of custom fitted AFO devices, each device within the series varying (as a whole) incrementally from one to the next with regard to the flexion angle.

In some embodiments, the one or more custom-fitted AFO components comprise a foot bed and an ankle cover. In some embodiments, fabricating the one or more associated custom-fitted AFO components includes a process of any of 3D printing or machining In some embodiments of the method of making a sequential series of AFO devices, each strut includes any of a thermoplastic carbon fiber composition or a thermoset resin. And in some embodiments of the method of making a sequential series of AFO devices, each of the one or more custom-fitted AFO components includes any of a thermoplastic carbon fiber composition or a thermoset resin.

Another aspect is directed to method of treating a patient to correct a pattern of idiopathic toe walking Embodiments of this method include acquiring a 3D digital profile of an ankle and foot of the patient in the form of an STL file; importing the STL file into a CAD application; and within the CAD application, creating a sequential series of individual pairs of digital 3D AFO models, each model including an incremental change in a flexion angle [of the ankle] compared to its neighbor within the series, said increment proceeding from an initial plantar flexion angle toward a dorsiflexion angle. Embodiments of the method continue with importing each model of the sequential series into an STL CAD manipulation application; fabricating the series of individual AFO devices, as directed by the series of individual 3D AFO models; and engaging the patient in a therapeutic regimen in which the patient wears one of each of the individual devices of the series for a period of time, moving from an initial device having the greatest degree of plantar flexion through the devices toward devices having diminishing angle of plantar flexion, and then having an increasing angle of dorsiflex.

### BRIEF DESCRIPTION OF THE FIGURES

FIGS. 1A and 1B are a lateral side and medial side views, respectively, of an ankle foot orthotic (AFO) device, according to one embodiment;

FIG. 2A shows a sequential series of individual AFO devices, in which (from left to right) a flexion angle moves incrementally, from one device to the next, from a plantar flexion configuration to a dorsiflexion configuration, according to one embodiment;

FIG. 2B shows two individual AFO devices: an initial device in a sequential series in a plantar flexion configuration and a final individual AFO device in the series in a dorsiflexion configuration, according to one embodiment;

FIG. 3 is a side view of an AFO device, according to an alternative embodiment;

FIGS. 4A-4E are perspective views illustrating a method of creating a custom ankle cover for an AFO device by way of molding a flat stock piece of thermoplastic material over a mold, according to one embodiment;

FIG. 5 is a side view of an AFO device disposed within a shoe, according to one embodiment;

FIGS. 6A and 6B are lateral perspective and medial perspective views, respectively, of an AFO device, according to an alternative embodiment;

FIG. 7 is a lateral perspective exploded view of the AFO device of FIGS. 6A and 6B;

FIG. 8A shows a sequential series of individual AFO devices, in which (from left to right) a flexion angle moves incrementally from a plantar flexion configuration to a dorsiflexion configuration, according to one embodiment;

FIG. 8B shows two individual AFO devices: an initial device in a sequential series in a plantar flexion configuration and a final individual AFO device in the series in a dorsiflexion configuration, according to one embodiment;

FIG. 9 is a side view of an AFO device disposed within a shoe, according to one embodiment;

FIG. 10 is a side view of an AFO device of Type B with an array of flexion angles shown for reference, according to one embodiment;

FIG. 11 is a side view of a foot with an array of plantar flexion and dorsiflexion angles shown for reference, according to one embodiment;

FIG. 12 is a flow diagram illustrating a method of fabricating a sequential series of orthopedic devices for a patient, according to one embodiment;

FIG. 13A is a schematic diagram illustrating a method of fabricating a sequential series of orthopedic devices for a patient, according to various alternative embodiments;

FIG. 13B is a schematic diagram illustrating a system for fabricating a sequential series of orthopedic devices for a patient, according to one embodiment;

FIG. 14 is a flow diagram illustrating a method of fabricating a sequential series of orthopedic devices for a patient, according to one embodiment;

FIG. 15 is a flow diagram illustrating a method of making a sequential series of AFO devices for a patient, the individual AFO devices varying incrementally in a flexion angle, according to one embodiment;

FIG. 16 is a flow diagram illustrating a method of making a sequential series of AFO devices for a patient, the individual AFO devices varying incrementally in a flexion angle, according to one embodiment;

FIG. 17 is a flow diagram illustrating a method of making a sequential series of AFO devices that vary incrementally from one to the next in a flexion angle, according to one embodiment; and

FIG. 18 is a flow diagram illustrating a method of making a sequential series of AFO devices for an individual patient, the individual AFO devices varying incrementally from one to the next in a flexion angle, according to one embodiment.

### DETAILED DESCRIPTION

Embodiments of the disclosed technology are directed toward orthopedic systems, devices, and methods that support correction of problematic neuromuscular patterns, skeletal deformities, and healing of broken or fractured bones by way of a sequential series of orthopedic devices that vary in form. The devices support and exert force on a targeted body part, including the bones and muscle within the body part. Healing bone breaks (as included in the scope of applying this technology) and correcting bone deformity can be seen as therapeutically distinct in various ways--both processes involve bone remodeling, and both rely, to varying degree, on supporting bone while exerting deliberately directed force. Altering problematic neuromuscular patterns, habits, or behaviors may also be subject to physical therapeutic intervention by sequential devices. All of these uses of a system of multiple orthopedic devices that vary incrementally in form may be understood broadly as reforming a body part from a presenting or pretreatment form or configuration toward a therapeutically desired form or configuration.

For simplicity, “orthopedic devices”, as used herein, will refer to any type of supportive or corrective orthopedic device that supports bone healing, the desirable correction of a deformity, or correcting a problematic neuromuscular pattern, habit, or behavior. Such devices, by way of example, may include casts, braces and/or splints. And such orthopedic devices may be applied to any body part that may be in need of such a device, such as, by way of example, limbs, extremities, or any portion of the axial skeleton. Typical embodiments of the orthopedic devices provided herein are “custom-fitted”, i.e., they are made specifically for an individual patient, and, accordingly, have dimensions and contours that are based on dimensions and contours of the body part of the patient for whom the orthopedic device is intended.

Custom fitting devices, per embodiments of the invention, may be arrived at by at least two approaches. In a first approach, the entire device is entirely custom made (made specifically for an individual patient, based on a digital profile of the relevant body portion). If it has multiple major components, all such components are custom made. In a second approach, custom fitting further includes the option of drawing components from an inventory that is diverse. By way of example, a device may have two major fitting components: one component being custom made, and the second component being drawn from a diverse inventory. The final product is nevertheless custom-fitted. In such a circumstance, typically the inventory-drawn component is relatively simple and corresponds to a relatively simple body parameter; the custom-made component is more complex and corresponds to a relatively complex body parameter. Diversity of the inventory simply refers to the range of available options. For example, an inventory of shirts that comes in small, medium, and large has relatively little diversity. An inventory of shirts that includes different collar sizes, chest sizes, sleeve lengths, and traditional fit or slim fit, has a diversity that provides more of a custom fit.

Casts and splints differ, in that casts are typically circumferentially complete, while splints typically have a longitudinally oriented separation that allows exposure to the underlying limb or body part. Casts are typically applied for a relatively long duration, while splints can be transiently removed and reapplied. In spite of the physical differences, their general therapeutic effect of body part support, protection, and immobilization are very similar. Braces are also broadly similar in terms of therapeutic effect, but in addition to hard, body-conforming pieces, braces also typically include soft good rigging and clasps that stabilize the hardware against the body. Selected examples of types of casts include thumb spica, short arm and long arm. Selected examples of splints for the upper body include sugar tong, ulnar gutter, thumb spica, finger, long arm posterior, and volar. Selected examples of splints for the lower body include knee splint, posterior leg splint, stirrup splint, and posterior leg splint combined with a stirrup splint. All of these preceding examples represent devices and conditions to which improvements associated with the disclosed technology could be applied.

Embodiments of the disclosed technology include a series of orthopedic devices that differ from each other incrementally through the series. Each succeeding device differs from its immediately preceding device in shape and/or dimension. Shape refers to any aspect of form, contouring, or angulation. These changes in shape or dimension are incremental and additive, leading efficiently toward the desired final physical form or neuromuscular pattern. Embodiments of the disclosed technology are also directed toward methods of making such systems and devices, as well as methods of healing a broken bone from an initially broken condition to a desired healed condition, by way of incrementally staged healing bone forms. Method embodiments also include methods of incremental correction of intact bone-based deformations.

The disclosed technology displaces a practice of making single orthopedic devices de novo, on an ad hoc basis, to address therapeutically directed orthopedic changes in dimension or shape that occur over time. The technology, instead, provides a sequential series of devices that support a controlled series of orthopedic changes over time. The series of devices, with their incremental changes in dimension and/or shape, in some instances, can be predetermined in terms of the devices and their timeline of use. In other instances, the dimensions and shapes of devices, and the timeline of use, can be made responsive to clinical particulars of the patient during the course of treatment. Whether predetermined or responsive to updated clinical input, what both paths have in common is a unity and continuity of device design and a rational and ordered progressive course toward a desired therapeutic result.

Embodiments of the technology may be directed to improving the range of motion in adults and children that have conditions of muscular tightness that seriously impede their ability to engage in activities of daily living. These conditions are currently addressed by methods generally known as serial casting. Accordingly, embodiments of the disclosed technology include an orthopedic device system for extending the range of motion in a body part (including one or more bones) from a range-limited condition toward a desired extended range of motion condition. In a more comprehensive expression of extending range of motion, underlying effects of the treatment are directed toward correcting joint alignment, as well as preventing a pathological course that would otherwise ensue, such as muscle and bone deterioration, and development of intractable deformity.

Such a system, accordingly, may include multiple, serially-organized, orthopedic devices, including an initial device and a final device, each device after the initial device representing a succeeding device to a preceding device, where each succeeding device varies from its preceding device in size and/or shape. Another way to describe such a sequential series of devices is that it includes an initial device and a final device, and one or more intervening devices. Typically, the initial device is configured to substantially fit the body part in its initially limited range of motion condition at a point near its range limit, and the final device is configured to direct the body part into the desired extended range of motion condition.

These features and aspects include a series of devices that vary through the series in size and/or shape. The series of devices may be manufactured by acquiring 3D data describing the targeted body part and applying one or more algorithms to drive the size and shape of the initial device toward the size and shape of the final device. These features and aspects may further include the use of 3D printing to fabricate devices directly, to fabricate positive molds around which to cast the orthopedic devices, or to fabricate negative molds for the devices.

Conditions associated with muscle tightness, immobility, and problematic neuromuscular patterns for which the technology is particularly applicable include scoliosis, cerebral palsy, spina bifida, brain injury, spinal cord injury, congenital abnormalities, muscular dystrophy, idiopathic toe walking, peripheral neuropathy, brachial plexus, arthrogryposis, and syndactyly. Patients may be children, adolescents, or adults. Children and adolescents are typically growing over the course of a treatment period, and accordingly, bones and body portions are gaining in dimension. All associated changes in body part dimension and shape may be accommodated by the sequentially ordered orthopedic devices, as disclosed, and such variables may be included in the algorithms applied to the sequential incremental changes in shape and/or dimension incorporated in each succeeding orthopedic device.

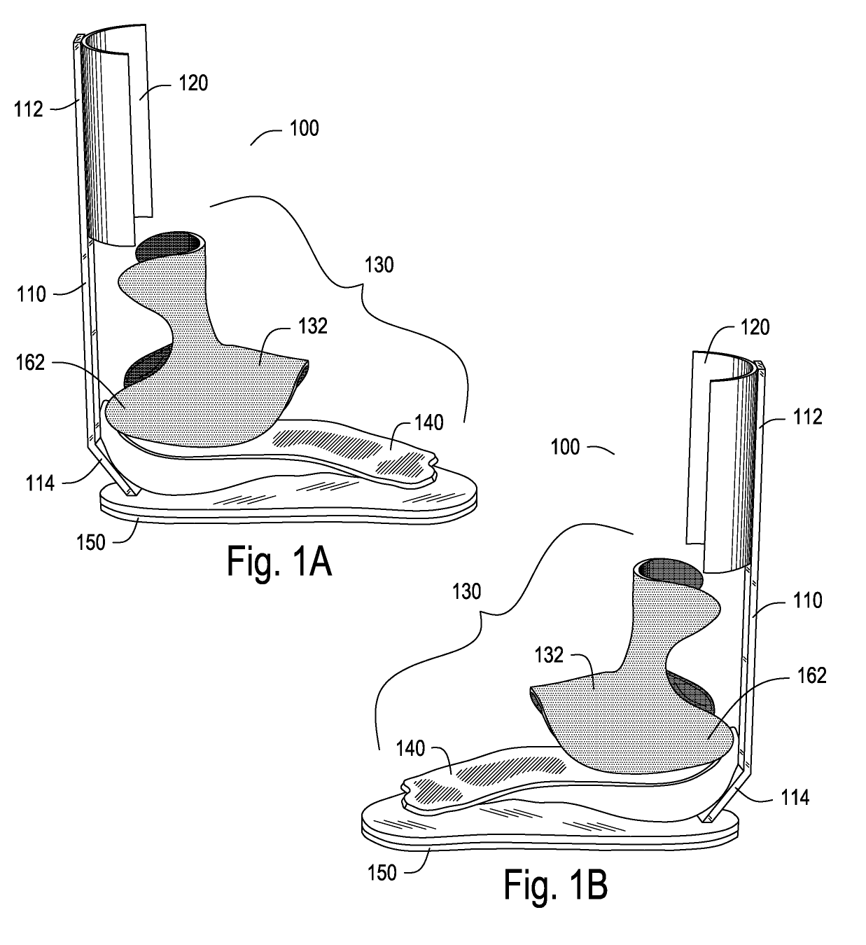

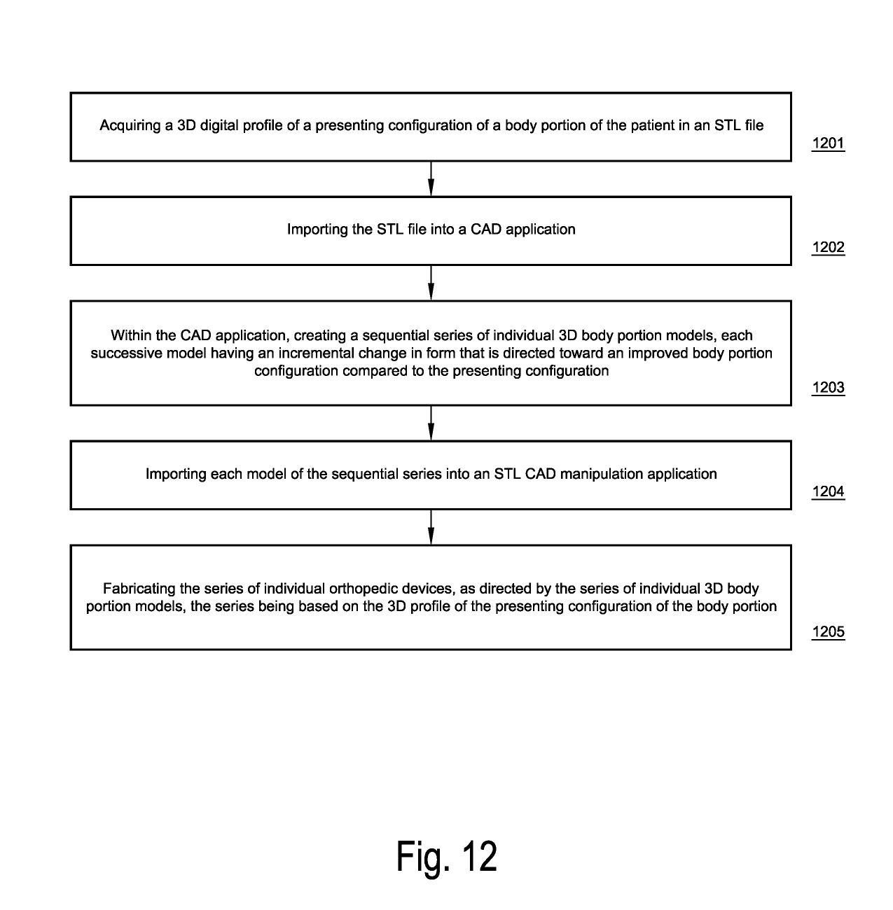

Various exemplary device and method embodiments are described below, in relation to FIGS. 1A-11 (devices) and FIGS. 12-17 (methods). These described exemplary embodiments are directed toward the treatment of idiopathic toe walking in pediatric patients. These embodiments, however, are provided for exemplary purposes only, to illustrate one possible application of the disclosed technology. In other embodiments, the devices and methods described herein may be applied to any of a number of other body parts and conditions, such as but not limited to those mentioned above. Therefore, the described embodiments should not be interpreted as limiting the scope of the present invention as it is defined in the claims.

In one embodiment, the technology includes a system of sequential orthopedic devices for facilitating healing of a broken bone from a broken condition to a desired final healed form. The system includes multiple serially organized orthopedic devices having an initial device and a final device, each device following the initial device representing a succeeding device to a preceding device. Each succeeding device varies from its preceding device in size and/or shape. The initial device is configured to fit to the body part with the broken bone in its initially injured or initially stabilized post-injury state. The final device is configured to support the bone in at least a partially healed state, and to direct healing of the bone toward the desired, final, healed form.

The initially broken condition of the bone includes any type of bone damage amenable to healing by way of being stabilized in a device. For example, a broken condition includes bone fractures, including non-union fractures. In some embodiments, multiple bones may be broken and in need of healing. Body parts that are typically appropriate for receiving an orthopedic device as described herein include the extremities—arms and hands, legs and feet—as well as portions of the axial skeletal system.

With regard to incremental and progressive variation in dimension or shape, embodiments of the multiple, serially organized, orthopedic devices may increase in size from an initially small dimension to a final large dimension. Such increases in size from an initially small dimension to a final large dimension may include incremental changes in dimension in the range of between about 0.1% to about 10% between the preceding device and the succeeding device. In particular embodiments, such dimensional changes may vary between about 0.25% to about 5% with respect to each other. Appropriate dimensions by which to size devices include any of a length, a nominal diameter, a cross-sectional area, and/or a volume. There is no absolute limit on the number of devices within a set of serially organized orthopedic devices, but typical examples of a series range between 2 devices and 20 devices. In particular examples, the number of devices in a series ranges between 3 devices and 12 devices.

In another aspect of incremental variation, the multiple serially organized orthopedic devices may vary with regard to an angular measure of a contoured aspect of the device. By way of example, the angular measure of a contoured aspect of the device can vary in the range of between about 0.1% to about 10% between the preceding device and the succeeding device. In particular embodiments, such shape changes may vary between about 0.25% to about 5% with respect to each other. The angular measure of a contoured aspect of the device can vary either by way of an increase or decrease in angular measure between the preceding device and the succeeding device.

Further, as noted above, orthopedic devices in a sequential series may also vary from preceding device to succeeding device with regard to both shape and dimension. The changes in shape and dimension may occur either coincidentally, in a closely linked manner, or sequentially or independently through the orthopedic device series. Shape changes and dimension changes can be plotted out to occur broadly over the same time course, but the rates of incremental change in shape and incremental change in dimension can be independent from each other. Further, in terms of the location within the device, the rates of change in shape or dimension may be spatially distributed. For example, if an orthopedic device has a distal end and a proximal end, shape changes can be localized within the distal end, proximal end, or in the center portion.

As noted above, embodiments of the technology may be directed to a system of sequential orthopedic devices for correcting a skeletal deformity. Some embodiments of the disclosed technology include an orthopedic device system for reforming a body part (including one or more bones) from a deformed condition to a desired final reformed condition. Such a system, accordingly, may include multiple, serially-organized, orthopedic devices, including an initial device, a final device, and one or more intermediate devices. Each device after the initial device may represent a succeeding device to a preceding device, where each succeeding device varies from its preceding device in size and/or shape. Typically, the initial device is configured to substantially fit the body part in its initially deformed condition or with a modest deviation toward a desired final reformed condition, and the final device is configured to direct the body part into the desired final reformed condition.

All of the features and aspects of the provided technology described above in the context of a series of orthopedic devices that are directed to supporting the healing of one or more broken bones through a series of devices apply to these embodiments as well, directed as they are to reforming a deformed body part. These features and aspects include a series of devices that vary through the series in size and/or shape, the basing of these sequential devices on acquisition of 3D data of the deformed body part, and applying one or more algorithms to drive the size and shape of the initial device toward the size and shape of the final device. These features and aspects further include the use of 3D printing to fabricate devices directly, to fabricate positive molds around which to cast the orthopedic devices, or to fabricate negative molds for the devices.

Deformed skeletal conditions for which the technology may be particularly applicable include scoliosis and club feet, by way of examples. Club feet are typically treated when the patient is an infant or child, in which case the treatment occurs over a time during which the feet and legs are growing. Scoliosis is a three-dimensional deformity of the spine that can present in infants, adolescents, and adults. Some occurrences of scoliosis are considered secondary to other primary conditions, but the majority of scoliosis cases are classified either as congenital or idiopathic. Surgical interventions are considered a last resort. Braces, including serial braces of various kinds, are the standard of care in all age ranges. In infants, children, and adolescents, the spine is still growing, plastic in nature, and thus amenable to reforming. The therapeutic objective of bracing is to reform the spine toward a more normal state.

In another aspect, embodiments of the technology may be directed toward facilitating a broken bone into a desired configuration. A method of healing a broken bone may include the following steps: (A) Supporting a body part hosting a broken bone in an initial orthopedic device, the initial device configured to support the bone in its initial broken condition or in a initially stabilized post-break condition; (B) Allowing the bone sufficient resident time in the initial orthopedic device to at least partially heal; (C) Removing the body part from the initial device; (D) Supporting the body part, now including the partially healed bone, in a succeeding device, the succeeding device varying in shape and/or dimension from the preceding initial device; (E) Repeating steps B, C, and D, in series, from preceding device to succeeding device, as necessary until the bone, supported in a final device, has healed into a desired final condition. At the conclusion of sufficient resident time in the final device, the method concludes by removing the body part from the final device.

In some embodiments, each of the multiple, serially organized, orthopedic devices may be formed by a 3D printing of a 3D digital profile based on acquisition of data from the broken bone in its initially injured state, or from bones that are not broken but are included in a body portion, such as a foot, that is affected by an undesirable presenting condition, as for example, the feet of a child presenting with idiopathic toe walking This approach to fabricating devices may be understood as a direct printing of the device, without any intermediary physical forms. The data for the 3D map of the broken or undesirably configured bone in its presenting state may be acquired by way of any of scanning, photographing, photogrammetry, mapping with a three-dimensional point reference device a three-dimensional digital or physical representation of the residual limb, imaging technologies, or by manual measurement. In particular, the imaging technologies may include any of magnetic resonance imaging (MRI), computed tomography (CT), ultrasound, X-ray imaging, positron emission tomography, microscopy imaging, and simulated image data. CT is an imaging method that has advantages of being fast and providing highly resolved 3D forms. MRI is also advantageous in some cases, because it can provide image data on soft tissue in addition to bone.

In some embodiments, each of the multiple, serially organized devices is formed by a 3D printing process. The timeline of actual manufacture of a set of serially organized devices may vary. By way of example, all of the multiple, serially organized devices may be formed by a 3D printing process in a single or substantially single printing session. In another example, each of the multiple serially organized devices may be formed by a 3D printing process in separate work sessions, on an as-needed basis.

In contrast to a direct printing of an orthopedic device, an alternative approach is to print a replicate of the affected body part, and then use that replicate as a positive mold upon which to cast the actual orthopedic device. Accordingly, in some embodiments, each of the multiple, serially-organized devices is formed by way of casting around a series of 3D printed positive models of the body part, the 3D map of the body part being created based on acquisition of data from the broken bone within the body part, the bone in its initially injured or initially stabilized post-injury state.

In yet another variation of the use of acquired 3D data and the fabrication of orthopedic devices as described herein, the 3D data may be used to form a negative mold of the orthopedic device. In these embodiments, the device is then fabricated by any suitable molding technique, such as pouring or injecting a flowable polymer into the mold, and allowing the device to set as it becomes the finished orthopedic device, or vacuum forming over a mold.

The variation in dimension and/or shape between a preceding device and a succeeding device may be determined by an algorithm that provides a step-by-step incremental path between the form of the initial device and the form of the final device. Such an algorithm provides a step-by-step path between each preceding device and its succeeding device, any of the size or shape of the succeeding device varying incrementally with respect to the preceding device, each succeeding device moving toward a configuration of the final device.

In one example, a broken bone may belong to a child in a rapid growth phase. Accordingly, the broken bone is also a growing bone, or a potentially growing bone, and the algorithm accordingly incorporates input that predicts a normal course of bone growth. Data input into the algorithm may include statistical predications of growth based on medical tables, the height and overall dimensions of the biological parents and close relatives, image data of epiphyseal growth zones to determine bone age and/or the like.

As noted above, embodiments of the disclosed technology include methods of making a system of multiple, serially organized, orthopedic devices that are used in a therapeutic regimen that directs reforming of a body portion from a presenting condition to a more favored condition. Two examples of such methods are disclosed. In a first example, the work product is a series of orthopedic devices. In a second example, the work product is a series of models of the body part that surrounds or supports the portion of the body that is being targeted for therapeutic reforming, the models serving as positive molds for creating the series of orthopedic devices.

Accordingly, in one example, such a method of making a set of serially organized orthopedic devices includes acquiring spatial data of the body part surrounding a broken bone, and in some embodiments, spatial data of the broken bone itself Based on these data, the method continues by applying an algorithm that plots a 3D course of bone form that evolves from that of the initially broken bone to that of a final desired form of the bone in a healed condition. The method continues by segmenting the 3D course of the evolving bone form into a set of discrete bone forms, and packaging the set of 3D bone forms into a data file readable by a 3D printer. The method then includes printing the set of data files to create a set of orthopedic devices corresponding to the discrete bone forms.

In a second example, in which the initial work product is a series of positive molds of the body part, the initial steps of the method are the same as the first example described above. This second exemplary method embodiment includes acquiring spatial data of the body part surrounding the broken bone, and preferably spatial data of the broken bone itself. Based on these data, the method continues by applying an algorithm that plots a 3D course of bone form that evolves from that of the initially broken bone to that of a final desired form of the bone in a healed condition. The method continues by segmenting the 3D course of the evolving bone form into a set of discrete bone forms, and packaging the set of 3D bone forms into a data file readable by a 3D printer. This method embodiment then includes printing the set of data files to create a set of model body parts corresponding to the discrete bone forms. Finally, the method involves using the set of model body parts as a set of positive molds, around which to cast a corresponding set of orthopedic devices. Embodiments of the technology include sequences of multiple, custom-fitted, orthopedic devices that vary incrementally from each other, one-to-next, in some particular aspect of form. The technology further includes embodiments of computer-implemented methods of making a sequential series of devices and computer-based systems that host and operate the appropriate software to transform a digital profile of a body part into a sequential series of models.

A sequential device series can also be understood in terms of a model that has a dynamic aspect that allows it to reshape (morph, reform, reconfigure) from an initial configuration (size and shape) to a second and preferred configuration. The dynamic aspect of the reconfiguration does not play out in a single adjustable device, but rather as a dynamic sequence embodied in a series of devices, in a flipbook manner. The configuration of the initial device in a series corresponds to the initial or presenting configuration of the relevant body portion of the patient. The configuration of the final device in a sequential series corresponds to the therapeutically desired final configuration of the relevant body portion. In terms of the flipbook analogy, the first page is the initial device, and the last page is the final device. The number of pages corresponds to the number of devices in the series. The rate at which the pages flip by corresponds to the rate at which a patient progresses through the devices.

Referring now to FIGS. 1A and 1B, an ankle foot orthotic (AFO) device 100 referred to herein as a “Type A” device, is illustrated in a lateral side view (FIG. 1A) and in a medial side view (FIG. 1B). In this embodiment, AFO device 100 includes a posterior strut 110 connected to a foot piece 130 by way of a fastener 162. Foot piece 130 includes a custom ankle cover 132 and a custom foot bed 140 that is supported by a foot bed platform 150. Fastener 162 connects ankle cover 132 and foot bed 140 together. Posterior strut 110 is a substantially vertical element, thereby establishing a vertical axis for reference, with a proximal portion 112 and a distal end 114. A calf support piece 120 may be attached to the proximal portion 112 of the posterior strut 110. Distal end 114 of posterior strut 110 is attached to foot piece 130, typically be way of foot bed 140. Fastener 162 (or “attachment”) may, in some embodiments, be easily detachable, to allow easy removal of ankle cover 132 from foot bed 140. Distal end 114 is disposed at an angle with respect to the generally vertical orientation of posterior strut 110 and further determinative of the angle of the posterior strut 110 with respect to the foot bed 140. This angle (an angle of flexion, and generally labeled herein as “αf”) and its significance are discussed further below.

In typical embodiments, foot piece 130 is custom fitted to an individual patient. Aspects of methods of custom fitting are described below. Although posterior strut 110 may be custom fitted to an individual patient, in typical embodiments it is available in a standard range of sizes and angulations (within distal end 114), and these variations may be included in an inventory, such that a custom fitting or custom fabrication is not necessary. Custom fitting, per embodiments of methods described herein, is based on acquiring a digital profile of the ankle and foot of the patient as the patient presents, prior to initiating treatment, and using that single initial digital profile as a source for modeling each of the AFO devices 100 (and AFO 200, as described below) in the sequential series of devices 100S (and 200S) that vary incrementally from device to device, through the full series of AFO devices.

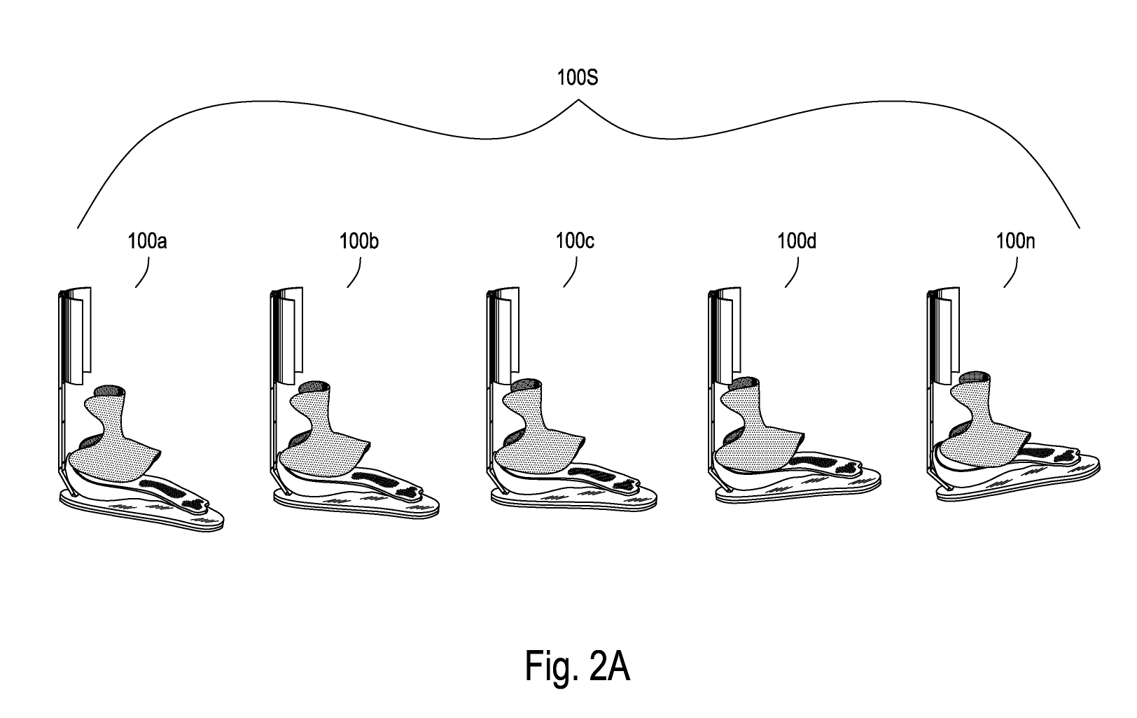

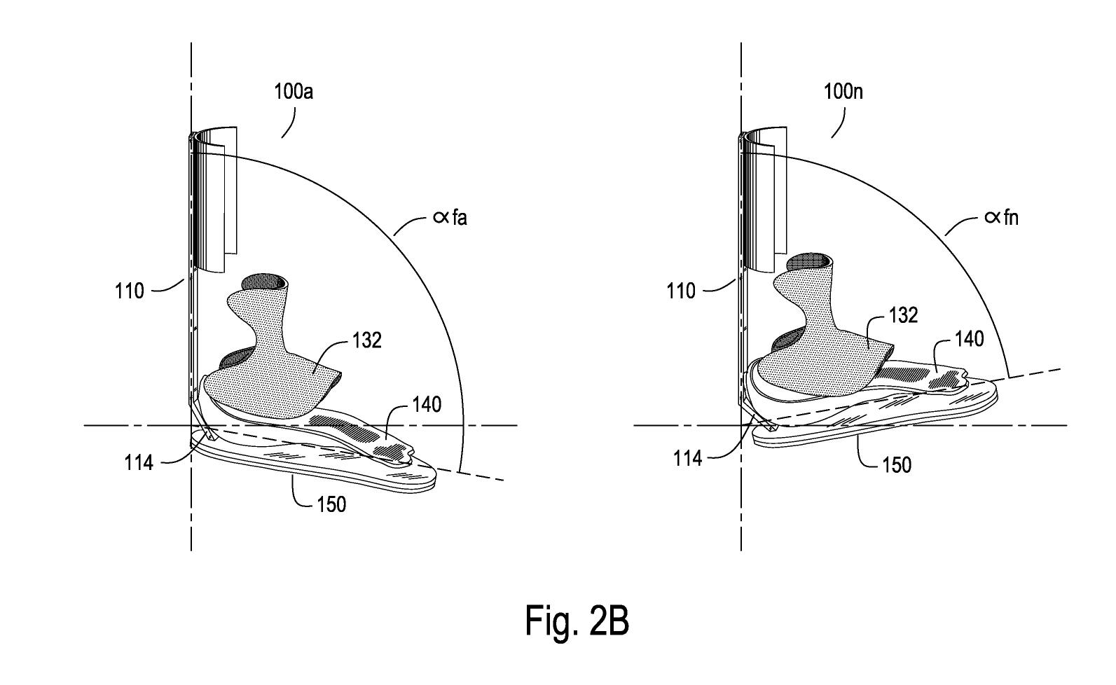

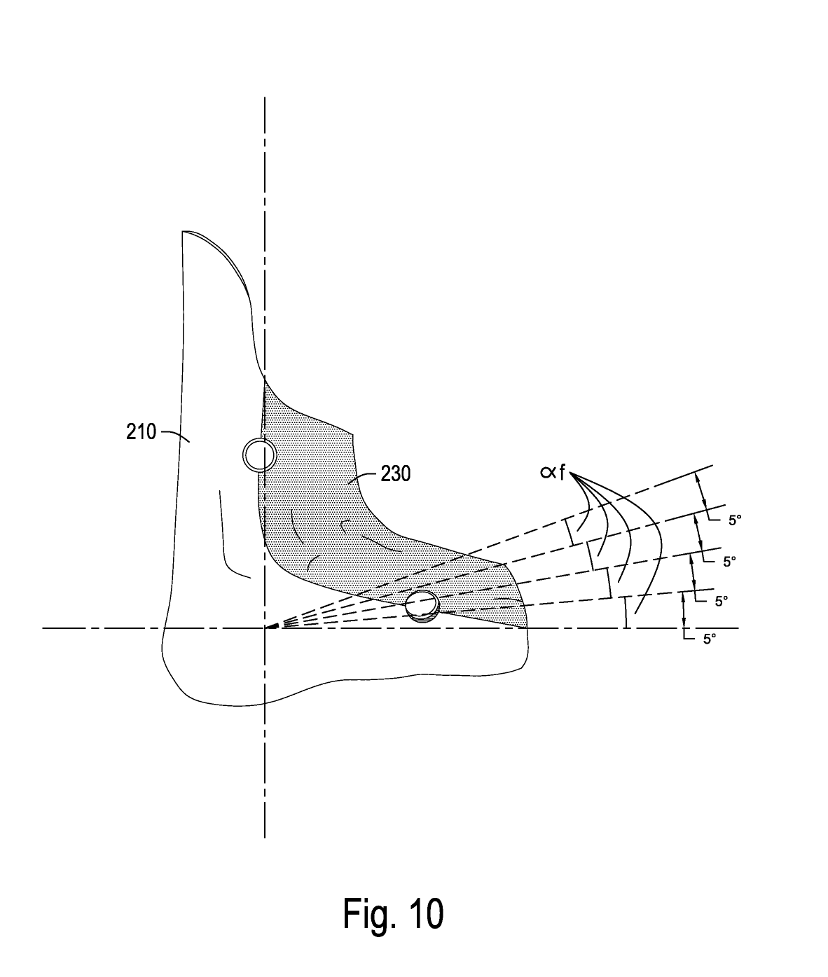

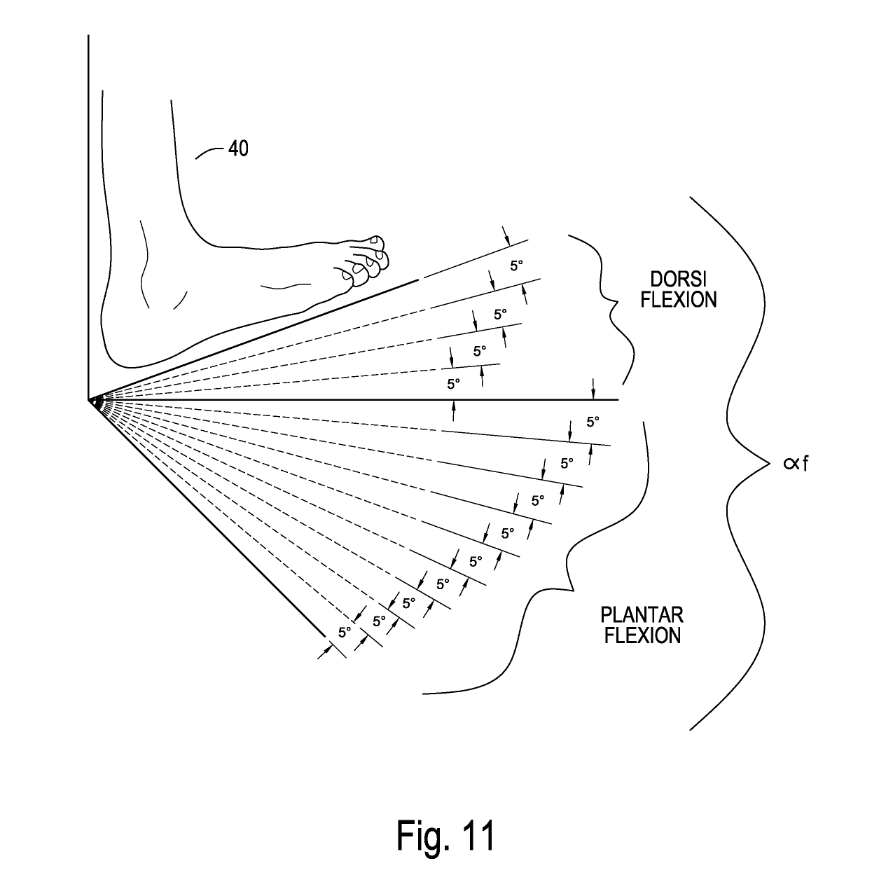

FIG. 2A shows a sequential series 100S of individual AFO devices of Type A (100a, 100b, 100c, 100d, . . . 100n) in which, from one device to the next, a flexion angle moves incrementally from a plantar flexion configuration to a dorsiflexion configuration. (Details of the nature of this change in angulation are shown in FIGS. 10 and 11). In a typical therapeutic regimen, a patient would initiate treatment by wearing a device 100a for a period of time, and then move on to devices 100b, then 100c, then 100d, and ultimately on to device 100n. The number of devices in a series 1005 may vary. In some particular examples, a series could include as few as two devices. In more typical examples, a series of sequential devices would range in number between three devices and about twelve devices.

The differences in flexion angle from one device to the next can vary in several ways. For example, nearest neighbor angle differences may typically range between about 1° and about 5°, but differences between nearest neighbor angles can be less than 1° and greater than 5°. Further, in typical embodiments, the difference in angulation from device to device is constant, for example, a constant incremental decrease in angle of 2°, or 4°. Further still, in some embodiments, the incremental change in angle from device to device need not be constant through the series. And further still, the incremental change in angle from device to device need not be predetermined at the outset of treatment, but can adjusted or determined by a physician during the course of treatment.

In some embodiments, the focus of a physician may not be on the incremental difference in ankle flexion angulation through the series, but rather on the total range of angle change that is desirable and the number of serial devices appropriate to achieve that angle. For example, the physician may estimate that a change in angle of 24° is desired over a series of eight devices. In this instance, a series of eight devices with an incremental difference in ankle angulation of 3° would be indicated.

The method of fabricating devices, as described herein, is very flexible. There is wide latitude in the number of devices in a sequential series and in the incremental differences in angle from device to device. These variables can be prescribed in a predetermined arrangement or can be customized to the patient per the clinical judgment of a physician.

Further still, a sequential series of devices can be fabricated during a single fabrication session at the outset of a therapeutic regimen, or individual devices within the series can be fabricated as needed over the course of a treatment. And further still, in an approach where a sequential series of devices is not fabricated at the outset of treatment, the degree of incremental angle change from one device to the next can be decided during the course of treatment, per the judgment of a physician or per the preference of a patient. Practicing clinical experience may eventually accumulate that can recommend particular angular increments, or particular rates of progression through devices. In some embodiments, the method may involve fabricating, for example, two or three devices, evaluating the patient's progress, and then, according to the patient's progress and the physician's evaluation, making decisions about angular increments and time intervals between devises going forward.

It is the digital nature of the method of fabricating these devices that allows this flexibility in approaches, while remaining cost effective. A patient does not need to be recast, per a conventional approach, in order to receive a next device in a series. And the incremental changes in angulation do not necessarily need to be predetermined, but can be adjusted while therapy is in progress. In the embodiments where devices are 3D printed, a turnaround time of 24-72 hours or even less may be achieved.

FIG. 2B shows two individual AFO devices of Type A: an initial device 100a in a sequential series (100S of FIG. 2A) in a plantar flexion configuration and a final individual AFO device 100n in the series in a dorsiflexion configuration. In this example, angle αfa of the AFO device 100a is about 105° from vertical (a plantar flexion angle of about 15°), while angle αfn of device 100n is about 80° from vertical (a dorsiflexion angle of about 10°).

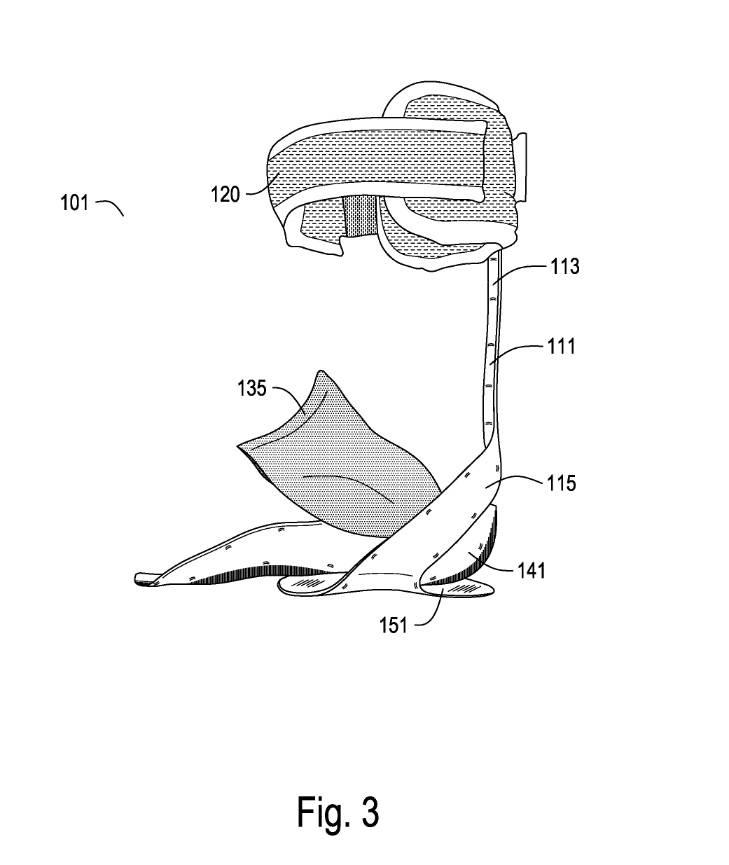

FIG. 3 shows an alternative embodiment of an AFO device 101, which differs from the embodiments depicted in in FIGS. 1A and 1B by virtue of an alternative configuration of a posterior strut 111. FIG. 3 shows ankle foot orthotic (AFO) device 101 in a lateral side view. AFO device 101 includes posterior strut 111 with an angled distal portion 115 that is connected to a custom ankle cover 135 and a custom foot bed 141, which is supported by a foot bed platform 151. A calf support piece 120 may be attached to the proximal portion 113 of posterior strut 111. Distal portion 115 is disposed at an angle with respect to the generally vertical orientation of posterior strut 111 and is determinative of the flexion angle of foot bed platform 151, and accordingly, of a patient's foot when the patient is wearing the device.

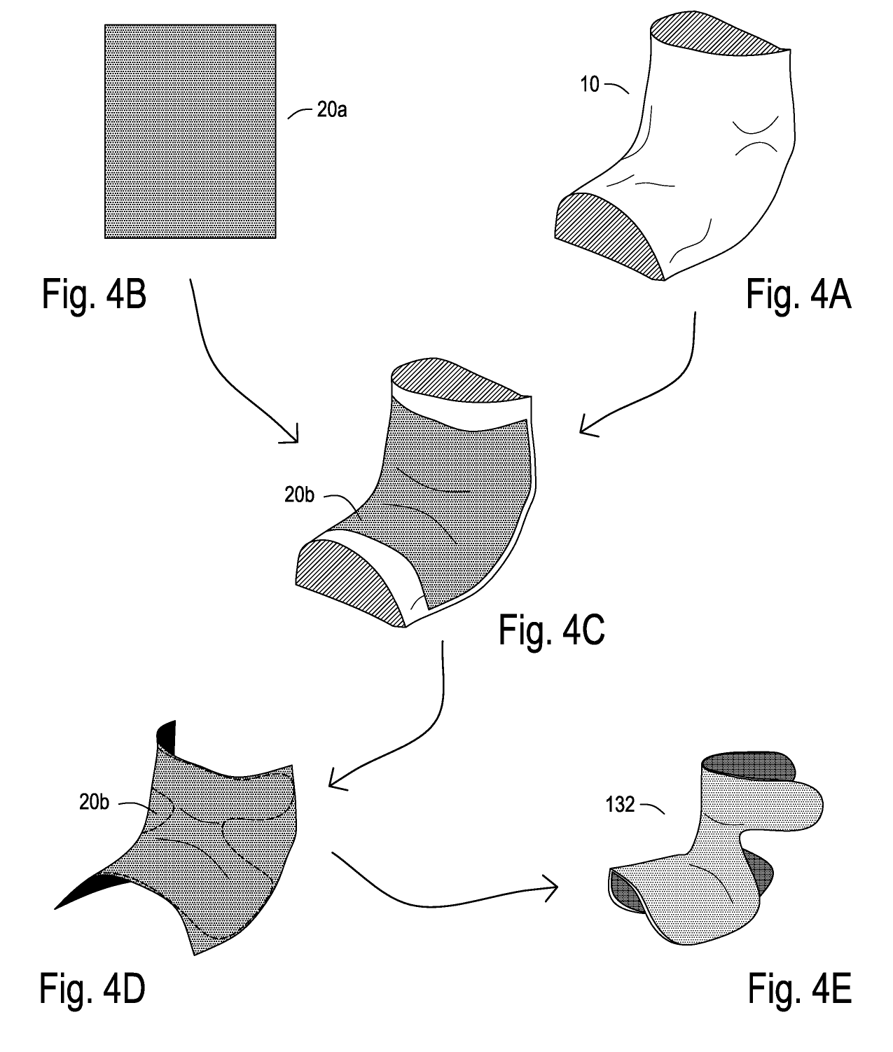

FIGS. 4A-4E schematically depict a method of creating a custom ankle cover 132 for a Type A device by way of molding a flat stock piece of thermoplastic material 20a over a mold 10. FIG. 4A shows a mold 10 of a portion of an individual patient's ankle FIG. 4B shows the flat stock piece of material 20a. FIG. 4C shows the stock piece 20b after having been heated and laid over the ankle portion mold. FIG. 4D shows the now-molded thermoplastic piece 20b with dotted lines where it is to be trimmed. FIG. 4E shows the completed ankle cover 132.

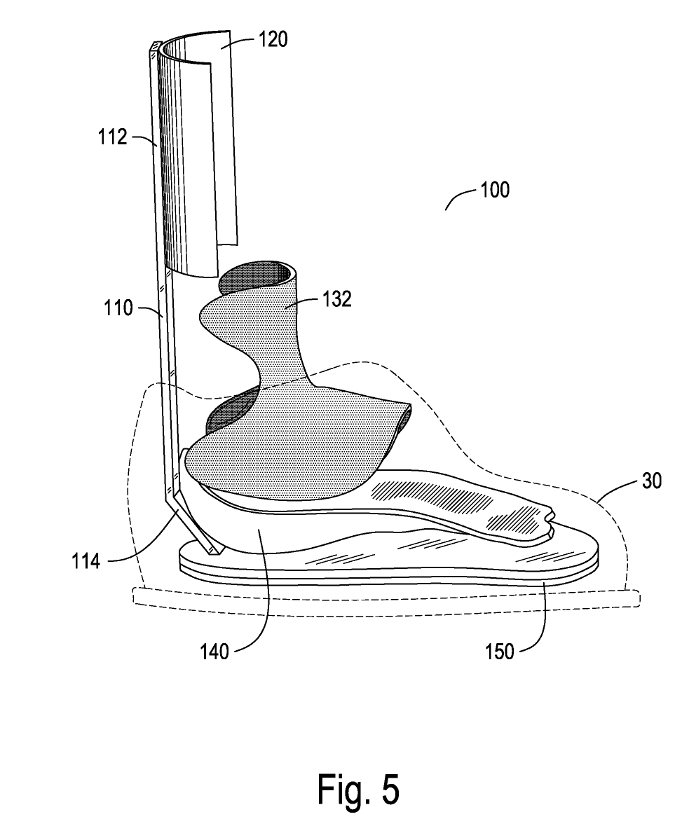

FIG. 5 shows a side view of AFO device 100 disposed within a shoe 30. Whether a patient wears a shoe over device 100 is a matter of personal preference, but typical embodiments of device 100 of Type A are designed with a low profile, such that wearing inside a shoe, perhaps a size larger than normal for the patient, is entirely feasible.

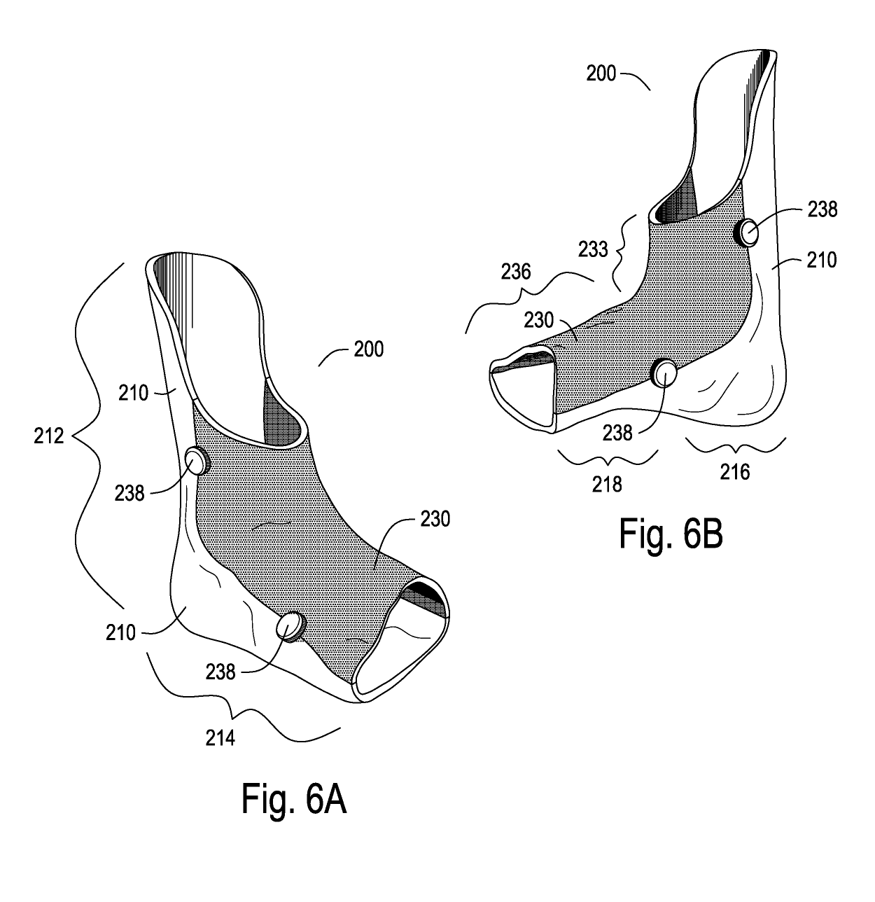

Referring now to FIGS. 6A and 6B, an alternative embodiment of an ankle foot orthotic (AFO) device 200, referred to herein as a “Type B” device, is shown in a lateral perspective view (FIG. 6A) and in a medial perspective view (FIG. 6B). AFO device 200 includes an integrated ankle-foot support portion 210 and an ankle cover portion 230. Both components (210 and 230) are fabricated by methods described below that yield a custom fit to an individual patient. Integrated ankle and foot support portion 210 includes an ankle portion 212 and a foot bed portion 214, which includes a heel portion 216 and an arch support portion 218 Ankle cover portion 230 includes a proximal ankle portion 233 and a foot dorsum portion 236. Integrated ankle-foot portion 210 and ankle cover portion 230 are fabricated based on the same patient-specific 3D model, and thus fit together seamlessly, and are connected by fasteners 238.

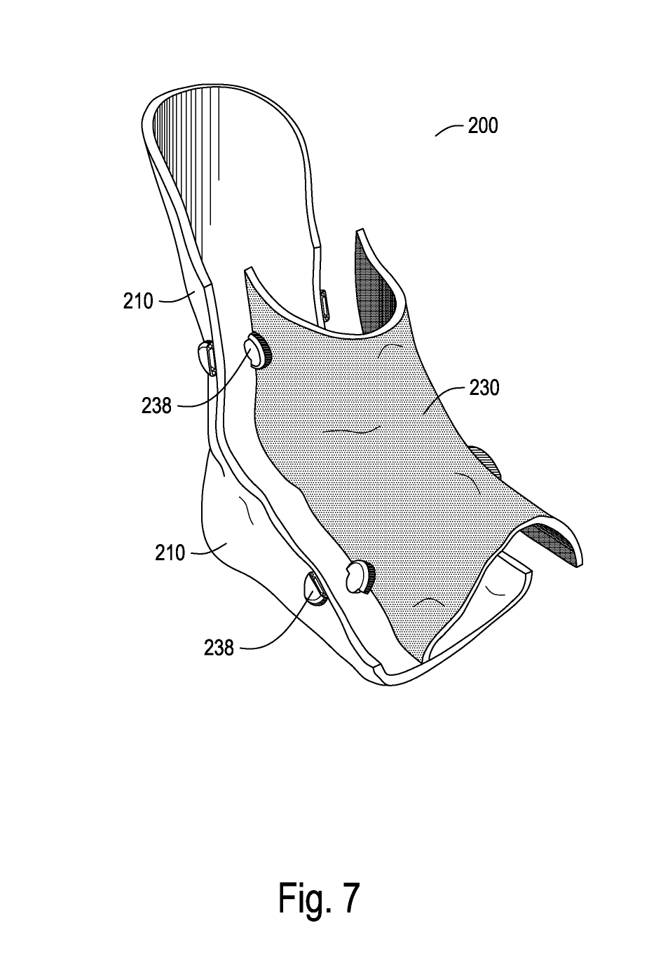

FIG. 7 is a perspective, exploded view of ankle foot orthotic (AFO) device 200,with ankle-foot support portion 210 and ankle cover portion 230 separated and spaced apart from each other.

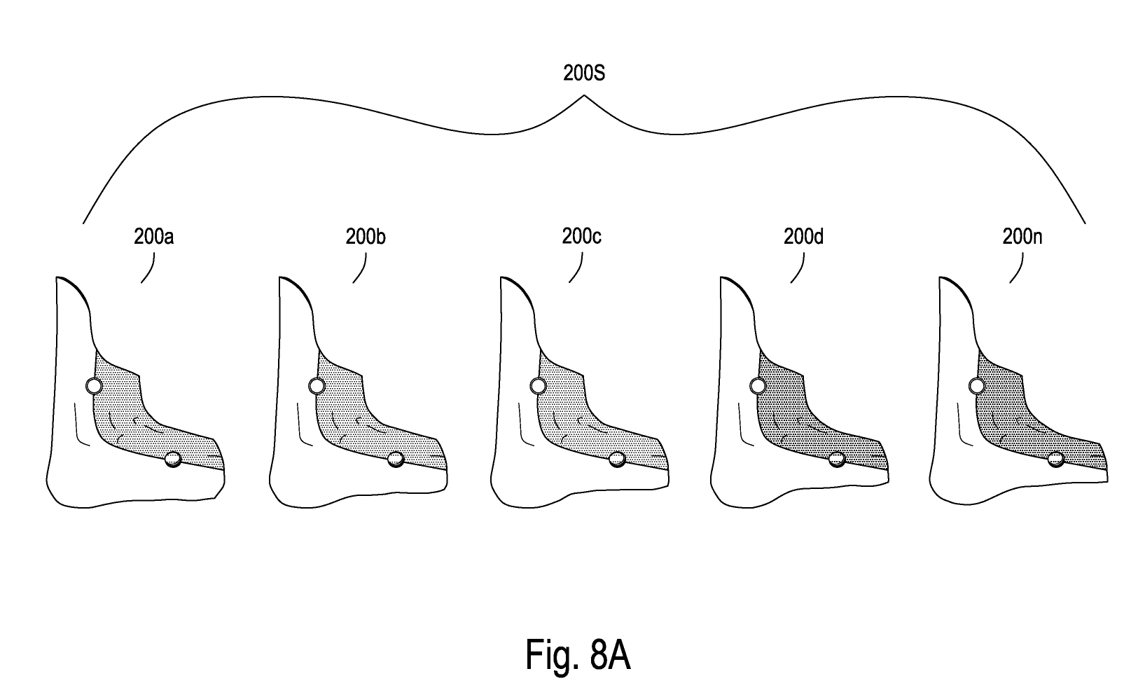

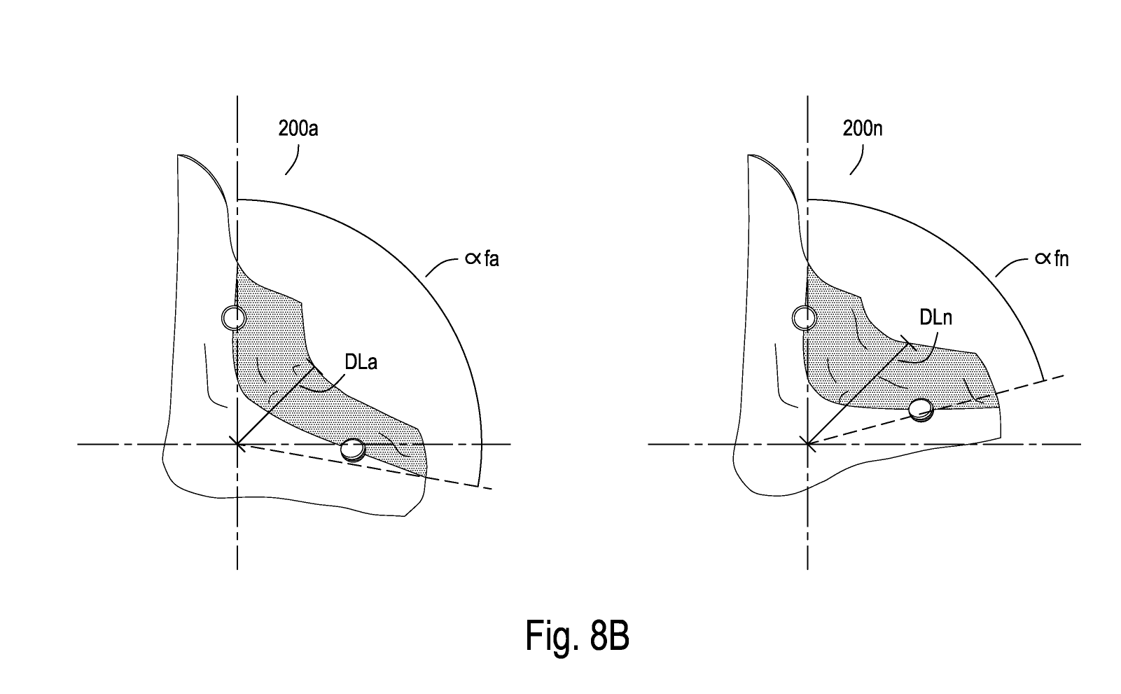

FIG. 8A shows a sequential series 200S of individual AFO devices of Type B (200a, 200b, 200c, 200d, . . . 200n) in which, from one device to the next, a flexion angle moves incrementally from a plantar flexion configuration to a dorsiflexion configuration. (Details of the nature of this change in angulation are shown in FIGS. 10 and 11). All of the considerations discussed regarding embodiments of Type A above in the context of in FIGS. 2A and 2B, apply here to sequential series 200S and constituent individual AFO devices of Type B (200a, 200b, . . . 200n).

FIG. 8B shows two individual AFO devices (200a and 200n) of Type B: an initial device 200a in sequential series 200S, in a plantar flexion configuration, and a final individual AFO device 200n of the series 200S, in a dorsiflexion configuration. In this example, angle of flexion αfa of AFO device 200a is about 105° from vertical (a plantar flexion angle of about) 15°), while angle of flexion αfn of device 200n is about 80° from vertical (a dorsiflexion angle of about 10°).

One consequence of a foot moving from plantar flexion to dorsiflexion is a thickening of the ankle profile across the anterior aspect of the ankle This follows simply from the volume of the ankle being constant, while the ankle configuration changes. Embodiments of the method of creating a series of sequential models of the foot accommodate this shape change, as indicated by the diagonal lines DLa and DLn (DLn being lengthened compared to DLa) respectively, marked across the central portion of the ankle of both devices (200a and 200n). This adjustment is a particular example of a consequence of a change in the positioning of a body portion, where such change creates a redistribution of body portion volume that may be accommodated in a serial modeling of a body portion undergoing an incremental change in form, as described herein.

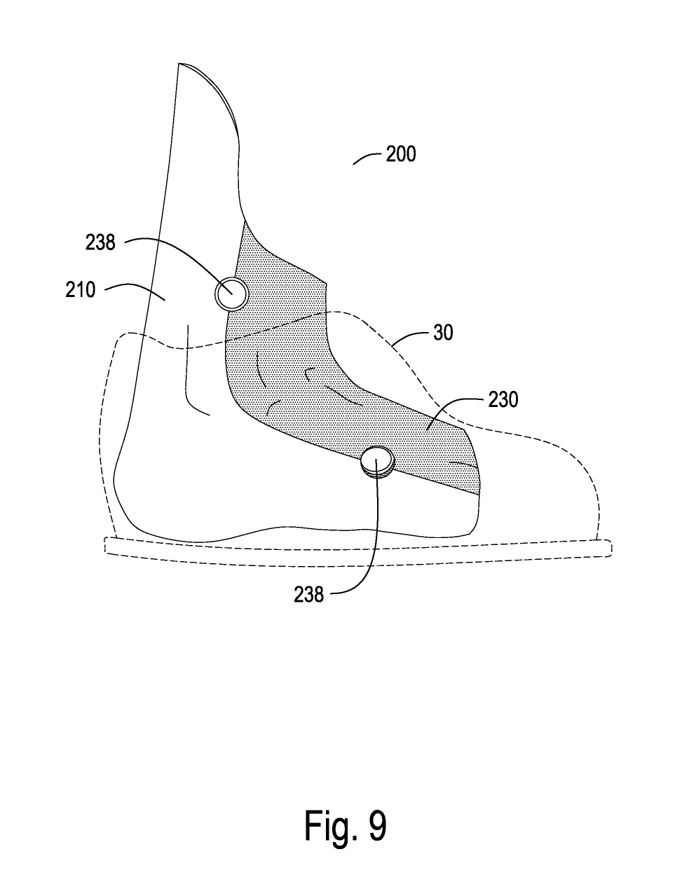

FIG. 9 shows a side view of AFO device 200 disposed within a shoe 30. Whether a patient wears a shoe over the device is a matter of personal preference, but typical embodiments of device 200 of Type B are designed with a low profile, such that wearing inside a shoe, perhaps a size larger than normal for the patient, is entirely feasible.

FIGS. 10 and 11 illustrate the orientation and magnitude of incremental changes in flexion angle of AFO device 200 through a sequential series of devices (FIG. 10) and of a foot as contained in such devices (FIG. 11). FIG. 10 is a side view of AFO device 200, with an array of flexion angles αf shown for reference. In this depiction, the vertical orientation of integrated ankle-foot support 210 is maintained as a constant, and various angles αf of the forward portion of the device, including portions of both ankle-foot support 210 and ankle cover 230, are schematically overlaid on device 200. In this example, incremental 5° angle changes are depicted. Although FIG. 10 shows an exemplary AFO device 200 of Type B, the figure would apply equally well if an AFO device 100 of Type A were used as the example instead.

FIG. 11 is a side view of a foot 40 with an array of plantar flexion and dorsiflexion angles shown for reference. In this example, a range of angles αf that range from a plantar flexion extreme of 45° (in 5° increments and a dorsiflexion angle of 20° in 5° increments) is shown. Foot 40 is shown in the orientation it would be within a final AFO device in a sequential series.

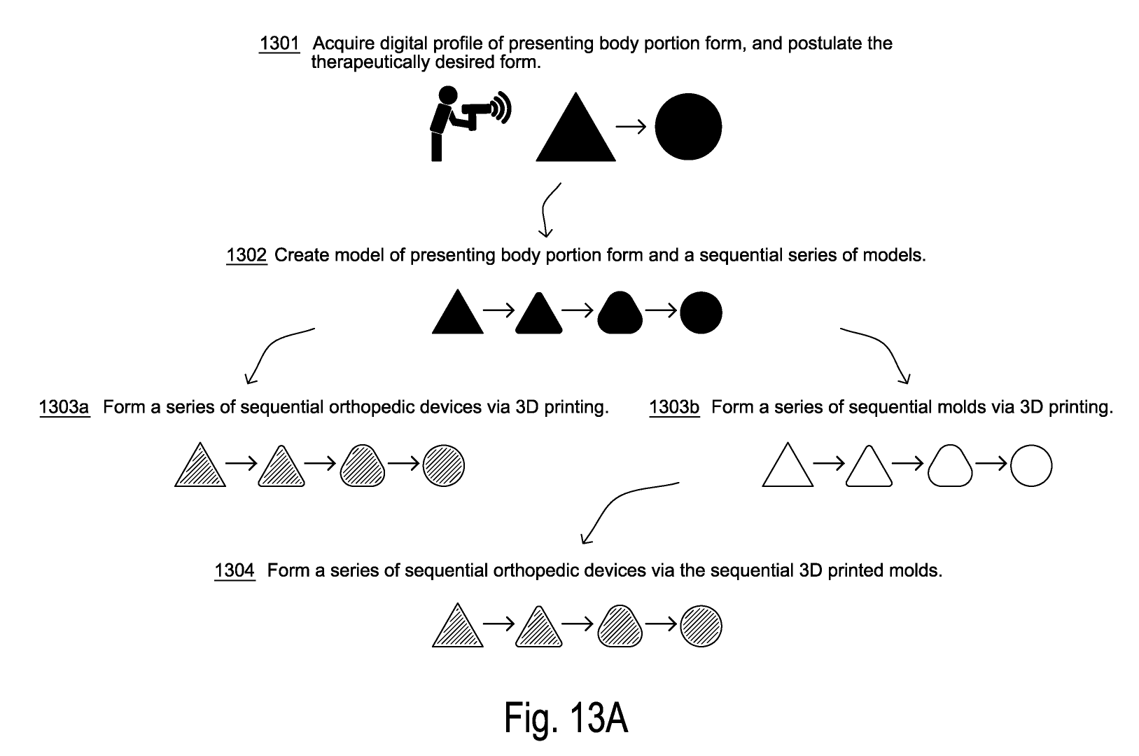

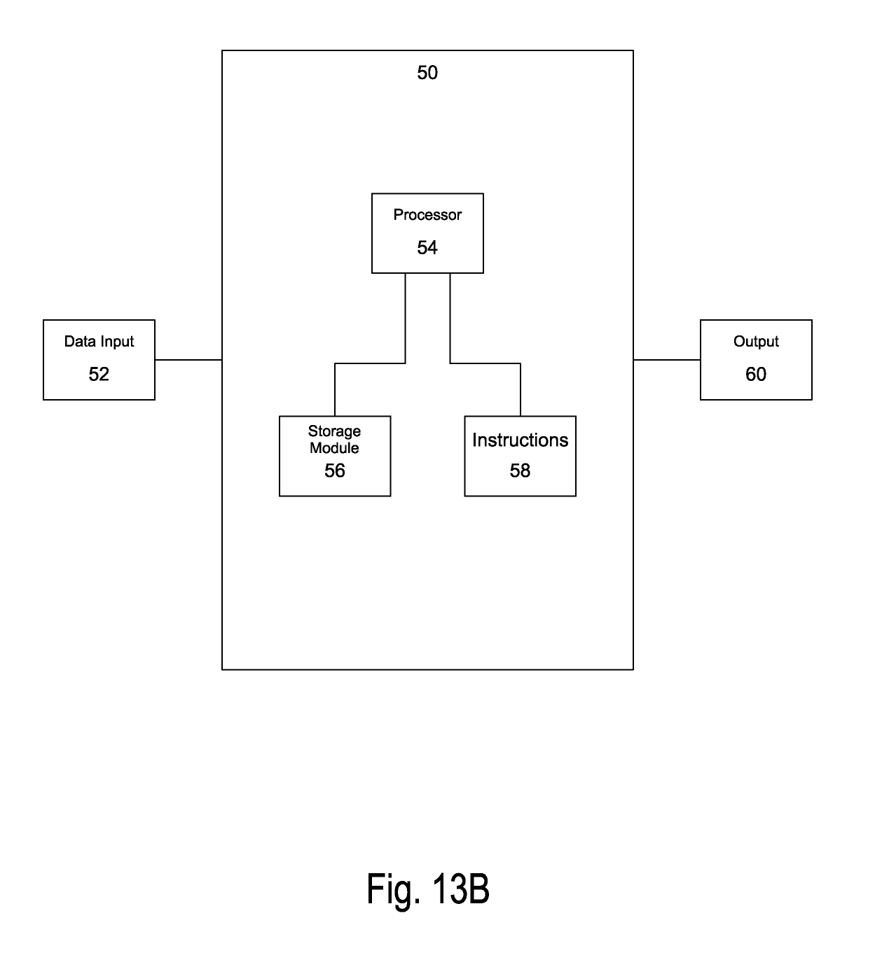

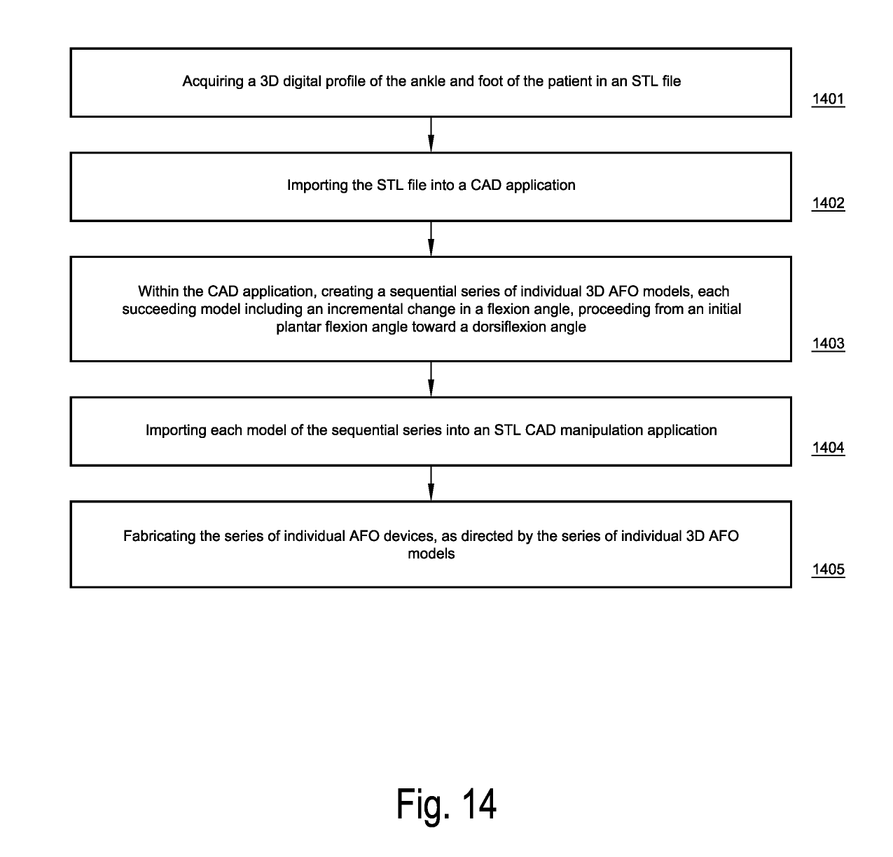

FIGS. 12-13B show methods and a system of making a series of sequential orthopedic devices that vary incrementally in form from one individual device to the next. FIGS. 14-18 illustrate a particular example of an orthopedic device and treatment plan in the form of a sequential series of AFO devices, in various embodiments.

Some characteristics and aspects of the methods described below may apply to multiple different embodiments. In some embodiments, for example, the described methods include making use of thermoplastic and thermoset materials for custom made AFO device components. Thermoplastic materials may include thermoplastic fiber composites, and such fiber may be in a substantially continuous form. In some embodiments, all of the fiber included within the thermoplastic composition is substantially continuous. With regard to the composition of the thermoplastic matrix, such composition may include a polymer matrix of polypropylene, polyethylene terephthalate (PET), acrylic, and/or polymethylmethacrylate (PMMA). Such AFO devices and components are typically fabricated based on a 3D digital model that is created from a 3D digital profile of a body portion (such as an ankle and foot) of a patient as the patient presents, at the outset of a treatment regimen. More particularly, from such a 3D digital model, an entire sequential series of AFO devices may be fabricated. Fabrication methods include direct fabrication from the 3D model by way of machining or 3D printing. In alternative fabrication methods, molds are created (typically by 3D printing) of each model in a sequential series, and then the devices or components are formed by way of these molds.

Some embodiments of the invention are directed to a method of fabricating a sequential series of orthopedic devices for a patient, the orthopedic devices varying incrementally in an aspect of form that moves progressively from a form that reflects a body portion of the patient as it presents at the outset of treatment toward a more favored form. Various steps of this method embodiment are recited below and shown in FIG. 12.
Step 1201 acquiring a 3D digital profile of a presenting configuration of a body portion of the patient in an STL file or functional equivalent.
Step 1202 importing the STL file into a CAD application.
Step 1203 within the CAD application, creating a sequential series of individual 3D body portion models, each successive model having an incremental change in form that is directed toward an improved body portion configuration compared to the presenting configuration.
Step 1204 importing each model of the sequential series into an STL CAD manipulation application.
Step 1205 fabricating the series of individual orthopedic devices, as directed by the series of individual 3D body portion models, the series being based on the 3D profile of the presenting configuration of the body portion.

FIG. 13A is a schematic diagram of a method of fabricating a sequential series of orthopedic devices for a patient, the orthopedic devices varying incrementally in an aspect of form. Embodiments of the method may be implanted in two versions, either as directed immediately to fabrication of a sequential series of devices or as directed to fabrication of a sequential series of devices by way of a sequential series of molds.

Steps toward direct fabrication of a series of sequential devices, in one embodiment, may include the following:
Step 1301 acquiring a 3D digital profile of a presenting configuration of a body portion of the patient;
Step 1302 creating an initial model of the presenting configuration of the body portion and a sequential series of models based on the initial model, the sequential models varying from each other in an aspect of form; and
Step 1303a by way of 3D printing, fabricating a sequential series of sequential orthopedic devices based on the sequential models.

Steps toward direct fabrication of a series of sequential devices by way of an intervening set of a series of sequential molds, in one embodiment, may include the following:
Step 1301 acquiring a 3D digital profile of a presenting configuration of a body portion of the patient;
Step 1302 creating an initial model of the presenting configuration of the body portion and a sequential series of models based on the initial model, the sequential models varying from each other in an aspect of form;

Step 1303b by way of 3D printing, creating a sequential series of sequential molds for orthopedic devices based on the sequential models; and
Step 1304 fabricating a series of individual orthopedic devices by way of molding devices from the series of sequential molds.

Turning now to Steps 1301-1304 in greater detail: in the top left corner of FIG. 13A is an abstract or pictographic depiction (a triangle) of a body portion of a patient in its presenting form, i.e., the body form of a patient as she or he first presents to the physician, prior to treatment beginning. Typically, the presenting body part form is medically problematic in some way. In the top right corner of FIG. 13A is, in part, a pictographic depiction (a circle) of the form of the body part that is desired for the patient, both by the patient and the physician. The progression from a triangular configuration to a circular configuration is purely representational of a therapeutic reshaping of a body part from a medically problematic configuration to a more favorable configuration. The stark configurational difference between the triangle and the circle represents any size, shape, or angular difference between a pretreatment body part configuration and the desired post-treatment configuration. The object of the therapeutic course (as guided by a sequential series of orthopedic devices with incremental changes in form) planned by the physician and patient is to reform the presenting configuration of the body part, moving it toward a more favorable configuration.

In Step 1301, a digital profile of a body portion in its presenting configuration is acquired. Any suitable method of acquiring a digital profile may be used, in various embodiments. Various approaches are enumerated above, including, merely by way of example, scanning, photogrammetry, MRI, and CT. In some embodiments, a single digital profile of the presenting body portion form is sufficient to drive the fabrication of a series of sequential orthopedic devices that vary incrementally in form until the final device, which is configured to be consistent with a final therapeutically desired configuration of the body portion.

In Step 1302, an initial model of the orthopedic device (based on the digital profile of the body portion in its presenting form or configuration) is created by a system 50 (see FIG. 13B). Following the creation of the initial model, system 50 then generates a sequential series of models that vary in form, and are sequentially directed to a therapeutically desired form or configuration (e.g., the circle of FIG. 13A). Incremental changes in form relate broadly to any parameter of dimension and/or shape, as schematically represented by the incremental progression in form from a triangle to circle. Variations in shape may include any aspect of contouring or angular relationship between or among vectors that can be assigned to structuralize body portions or devices within the sequential series of orthopedic devices.

In Step 1303A, a series of devices are fabricated from the sequential series of device models. Methods may include any of carving, machining, or 3D printing. In comparison to Step 1304, below, which uses molds, Step 1303A may be considered to be a direct fabrication (i.e., directly from model to device). 3D printing technology is developing quickly and moving into many different practical applications. 3D printed materials or media include a wide range of plastics, metals, and earthenware. 3D printable metals include, by way of example, platinum, gold, silver, brass, bronze, and steel. Among plastics, nylon or polyamide may be particularly suitable for devices, because it is lightweight and strong.

Other 3D-printable materials may be particularly appropriate for printing molds, such as “sandstone”, a ceramic that is combined with plaster of Paris, by way of a “Zcorp” process. Hardening agents can be added to the 3D print media or coated on an article after printing, which hardens the 3D-printed surface, and further provides a level of heat resistance that is advantageous molds. In yet another option, some 3D printing systems use paper. In this approach, sheets of paper are cut per a 3D CAD file, and each layer of paper is adhered to the one before it. The final piece is hard and dense. The 3D-printed article may also be post-processed with a liquid resin hardener (such as epoxy), and it can then be used as a mold.

In Step 1303B, a series of molds are fabricated from the sequential series of device models. Methods may include any of carving, machining, or 3D printing. Step 1303B may be considered to be an indirect or preliminary first step in fabrication of a sequential series of orthopedic devices

In Step 1304, a sequential series of orthopedic devices is fabricated by way of the sequential series of molds created in Step 1303B. Notably, the devices created by Step 1304 are substantially identical to the devices created by Step 1303A.

Any method described or depicted herein (FIGS. 12-18) may be embodied within a computer-implemented system. FIG. 13B is a schematic diagram of a system 50 for providing a sequential series of orthopedic devices (e.g., 100S or 200S) for a patient, the individual devices in the series varying incrementally in form from one to the next. System 50 is configured to operate the various steps of the schematic flow diagram shown in FIG. 13A.

Input 52 to system 50 includes a digital profile of at least a portion of a body part of patient in a presenting configuration (as represented by the triangle of FIG. 13A), per Step 1301 of FIG. 13A. Other types of input may include specifications associated with the particular orthopedic device to be fabricated. Input may further include instructions from the patient's physician, such instructions including dimensional or angular ranges between sequential devices, or between the initial device and the final device. Data input 52 may be stored in storage module 56, and acted upon by instructions 58 placed in the system 50, all activity being controlled and coordinated by processor 54. By processing input, information held in storage module 56 per instructions 58, an output 60 is generated.

Output 60, per embodiments of the invention, is typically a series of orthopedic device models which vary incrementally in some particular aspect of form, the first device model within the sequential series being sized and configured for the body portion of the patient in its presenting form, as acquired in Step 1301 of FIG. 13A. From that first model, the subsequent models move progressively toward the more favored configuration of the body portion (as represented by the circle of FIG. 13A). Sequential models (as shown, for example, in FIGS. 2A and 8A) may include a single unitary device, or a device with one or more component pieces, as well as left-right paired models).

Output 60, in the form of a sequential series of orthopedic device models, per embodiments of the invention, may be directed toward operation of machining devices, carvers, or 3D printers. Articles fabricated by any of these approaches may include a series of orthopedic devices, or a series of molds from which such a series of orthopedic devices may be fabricated.

The preceding description of orthopedic devices, arranged as a sequential series of devices that vary in form may be applied to many types of orthopedic devices, such as casts, splints, and braces, as enumerated above.

FIGS. 14-18 relate to methods for making a series of ankle-foot orthotic (AFO) devices—just one example of an orthopedic device for which a sequential series of such devices may deliver therapeutic benefit. Accordingly, some embodiments of the invention are directed to a method of making a sequential series of (AFO) devices for a patient, the individual AFO devices varying incrementally in a flexion angle at a site corresponding to the patient's ankle As mentioned several times previously, the example of a series of AFO devices and methods for making AFO devices are provided for exemplary purposes only. The methods and systems described herein may be applied to any other suitable splint, brace, cast device or other orthopedic devices in alternative embodiments.

Referring now to FIG. 14, in one embodiment, a method for making a sequential series of AFO devices may include the following:
Step 1401 acquiring a 3D digital profile of the ankle and foot of the patient in an STL file or functional equivalent;
Step 1402 importing the STL file into a CAD application;
Step 1403 within the CAD application, creating a sequential series of individual 3D AFO models, each succeeding model including an incremental change in a flexion angle, proceeding from an initial plantar flexion angle toward a dorsiflexion angle;
Step 1404 importing each model of the sequential series into an STL CAD manipulation application; and
Step 1405 fabricating the series of individual AFO devices, as directed by the series of individual 3D AFO models.

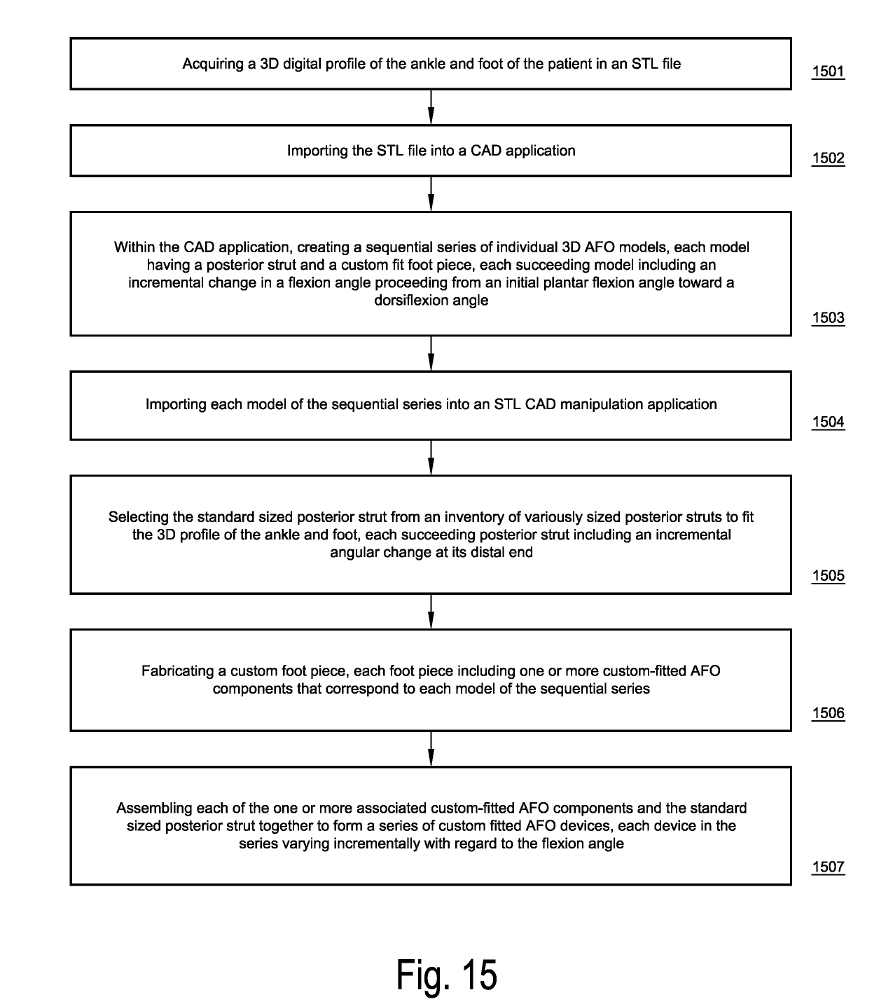

As shown in FIG. 15, some embodiments of the invention are directed to a method of making a sequential series of AFO devices for a patient, the individual AFO devices varying incrementally in a flexion angle. In one embodiment, the method may include the following steps:
Step 1501 acquiring a 3D digital profile of the ankle and foot of the patient in an STL file or functional equivalent;
Step 1502 importing the STL file into a CAD application;
Step 1503 within the CAD application, creating a sequential series of individual 3D AFO models, each model having a posterior strut and a custom fit foot piece, each succeeding model including an incremental change in a flexion angle proceeding from an initial plantar flexion angle toward a dorsiflexion angle;
Step 1504 importing each model of the sequential series into an STL CAD manipulation application;
Step 1505 selecting the standard sized posterior strut from an inventory of variously sized posterior struts to fit the 3D profile of the ankle and foot, each succeeding posterior strut including an incremental angular change at its distal end;
Step 1506 fabricating a custom foot piece, each foot piece including one or more custom-fitted AFO components that correspond to each model of the sequential series; and
Step 1507 assembling each of the one or more associated custom-fitted AFO components and the standard sized posterior strut together to form a series of custom fitted AFO devices, each device in the series varying incrementally with regard to the flexion angle.

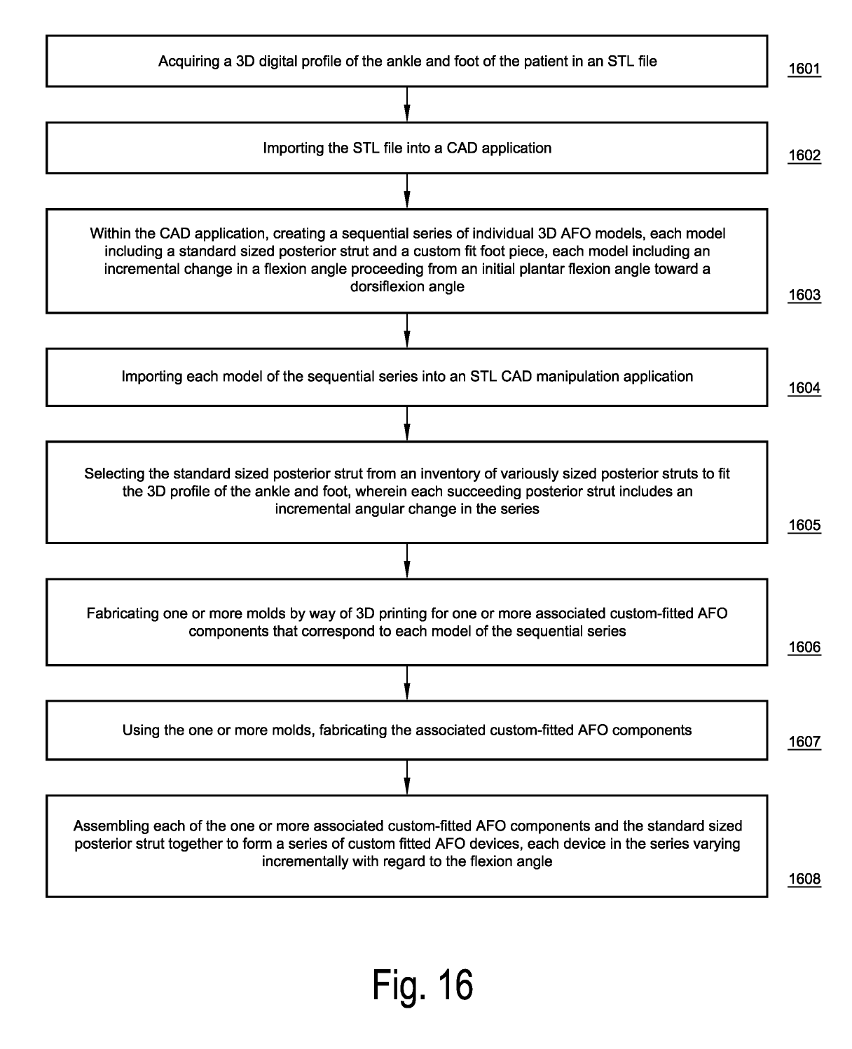

As shown in FIG. 16, some embodiments of the invention are directed to a method of making a sequential series of AFO devices for a patient, where the method involves using molds and the individual AFO devices vary incrementally in a flexion angle. In one embodiment, the method may include the following steps:
Step 1601 acquiring a 3D digital profile of the ankle and foot of the patient in an STL file or functional equivalent;
Step 1602 importing the STL file into a CAD application;
Step 1603 within the CAD application, creating a sequential series of individual 3D AFO models, each model including a standard sized posterior strut and a custom fit foot piece, each model including an incremental change in a flexion angle proceeding from an initial plantar flexion angle toward a dorsiflexion angle;
Step 1604 importing each model of the sequential series into an STL CAD manipulation application;
Step 1605 selecting the standard sized posterior strut from an inventory of variously sized posterior struts to fit the 3D profile of the ankle and foot, wherein each succeeding posterior strut includes an incremental angular change in the series;
Step 1606 fabricating one or more molds by way of 3D printing for one or more associated custom-fitted AFO components that correspond to each model of the sequential series;
Step 1607 using the one or more molds, fabricating the associated custom-fitted AFO components; and
Step 1608 assembling each of the one or more associated custom-fitted AFO components and the standard sized posterior strut together to form a series of custom fitted AFO devices, each device in the series varying incrementally with regard to the flexion angle.

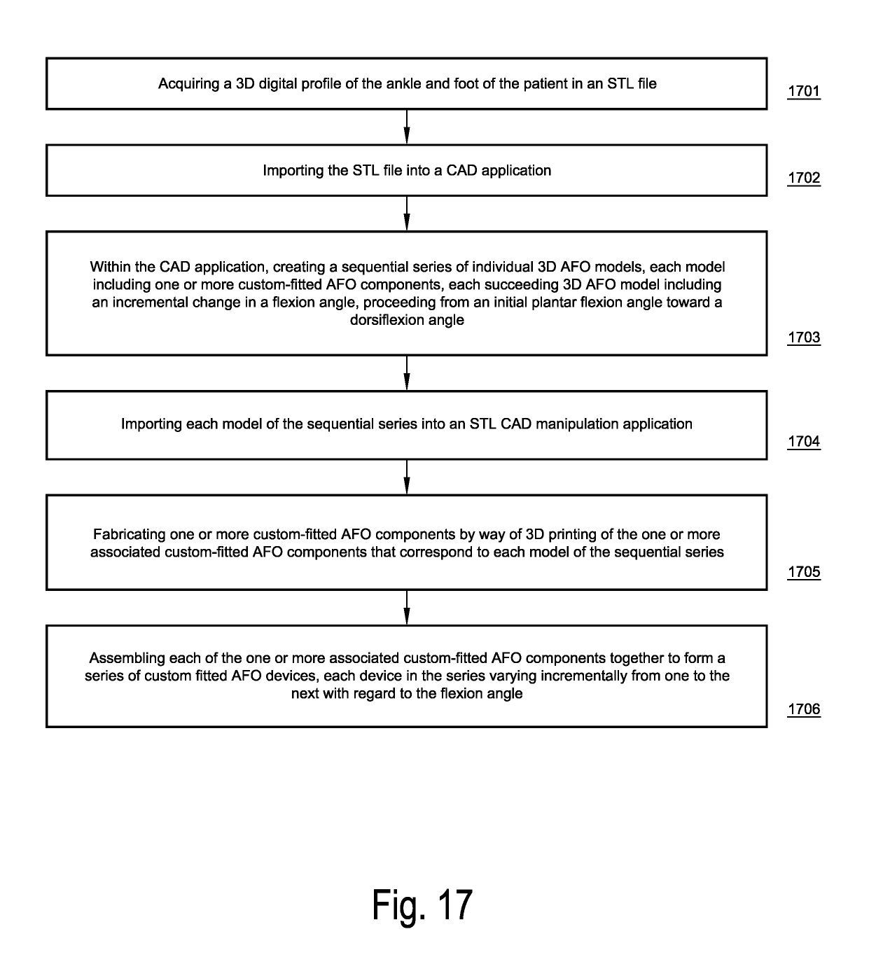

As shown in FIG. 17, some embodiments of the invention are directed to a method of making a sequential series of AFO devices that vary incrementally from one to the next in a flexion angle. In one embodiment, the method includes the following steps:
Step 1701 acquiring a 3D digital profile of the ankle and foot of the patient in the form of an STL file or functional equivalent;
Step 1702 importing the STL file into a CAD application;
Step 1703 within the CAD application, creating a sequential series of individual 3D AFO models, each model including one or more custom-fitted AFO components, each succeeding 3D AFO model including an incremental change in a flexion angle, proceeding from an initial plantar flexion angle toward a dorsiflexion angle;
Step 1704 importing each model of the sequential series into an STL CAD manipulation application;
Step 1705 fabricating one or custom-fitted AFO components by way of 3D printing of the one or more associated custom-fitted AFO components that correspond to each model of the sequential series; and
Step 1706 assembling each of the one or more associated custom-fitted AFO components together to form a series of custom fitted AFO devices, each device in the series varying incrementally from one to the next with regard to the flexion angle.

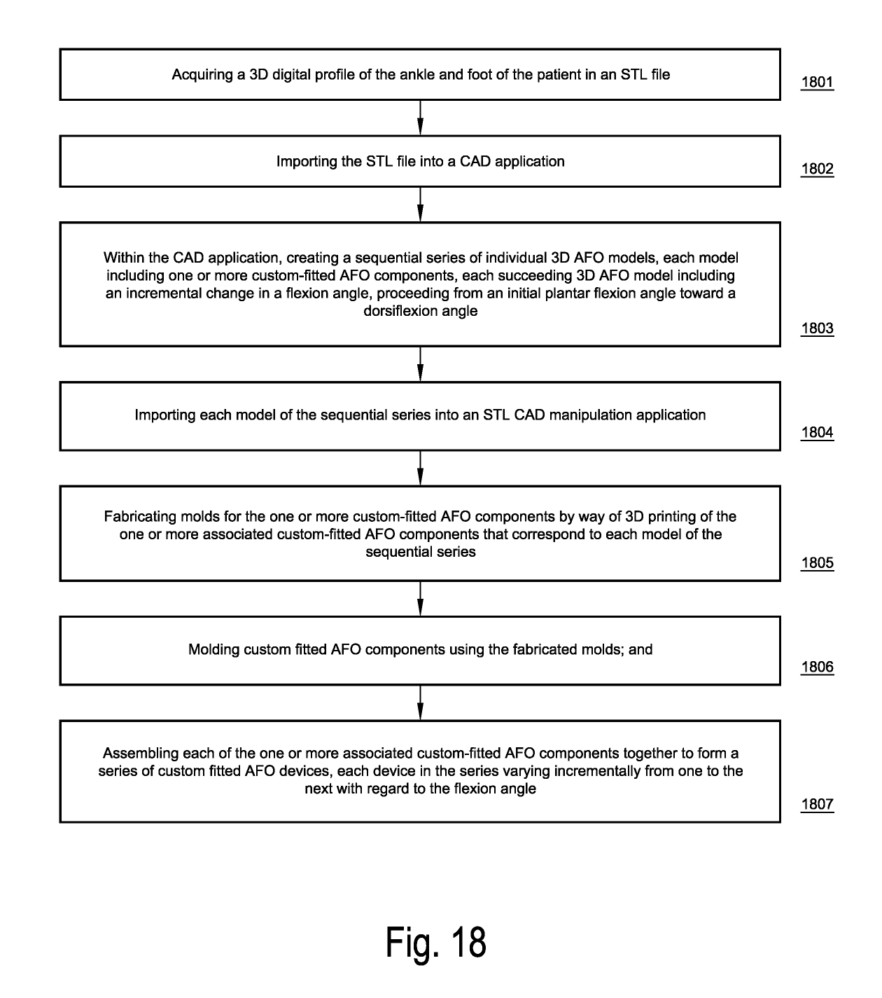

As shown in FIG. 18, some embodiments of the invention are directed to a method of making a sequential series of AFO devices for an individual patient, the individual AFO devices varying incrementally from one to the next in a flexion angle. In one embodiment, the method includes the following steps:
Step 1801 acquiring a 3D digital profile of the ankle and foot of the patient in the form of an STL file;
Step 1802 importing the STL file into a CAD application;
Step 1803 within the CAD application, creating a sequential series of individual 3D AFO models, each model including one or more custom-fitted AFO components, each succeeding 3D AFO model including an incremental change in a flexion angle, proceeding from an initial plantar flexion angle toward a dorsiflexion angle;
Step 1804 importing the model of the sequential series into an STL CAD manipulation application;
Step 1805 fabricating molds for the one or custom-fitted AFO components by way of 3D printing for one or more associated custom-fitted AFO components that correspond to each model of the sequential series;
Step 1806 molding custom-fitted AFO components using the fabricated molds; and
Step 1807 assembling each of the one or more associated custom-fitted AFO components together to form a series of custom fitted AFO devices, each device in the series varying incrementally from one to the next with regard to the flexion angle.

One aspect of the invention is directed to a method of treating a patient to correct a pattern of idiopathic toe walking—a condition that occurs particularly in pediatric patients. Embodiments of this method include acquiring a 3D digital profile of an ankle and foot of the patient in the form of an STL file; importing the STL file into a CAD application; and within the CAD application, creating a sequential series of individual pairs of digital 3D AFO models, each model including an incremental change in a flexion angle of the ankle compared to its neighbor within the series, the increment proceeding from an initial plantar flexion angle toward a dorsiflexion angle. Embodiments of the method continue with importing each model of the sequential series into an STL CAD manipulation application; fabricating the series of individual AFO devices, as directed by the series of individual 3D AFO models; and engaging the patient in a therapeutic regimen in which the patient wears one of each of the individual devices of the series for a period of time, the patient moving from an initial device having the greatest degree of plantar flexion through the devices toward devices having diminishing angle of plantar flexion, and then having an increasing angle of dorsiflexion.

In some embodiments, the 3D digital profile of the ankle and foot needs to be acquired once, and from that profile a series of AFO devices can be created that allow completion of a course of therapy. In some instances, it may become evident, either to the patient or the physician, that the ankle and foot of the patient are deviating from what was expected to be a straightforward therapeutic change in form. In this instance, a second 3D digital profile of the ankle and foot can be acquired, and a course of therapy with a reset series of sequential devices can by embarked on.

Any one or more features of any embodiment described herein (e.g., a sequential series of devices, any individual device, or any method of making or using the invention) may be combined with any one or more other features of any other embodiment, without departing from the scope of the invention. Further, the invention is not limited to the embodiments that are described or depicted herein for purposes of exemplification, but is to be defined only by a fair reading of claims appended to the patent application, including the full range of equivalency to which each element thereof is entitled. Further, while some theoretical considerations have been offered to provide an understanding of the technology (e.g., the effectiveness of a therapeutic regimen for a patient using an embodiment of the invention), the claims are not bound by such theory.

What is claimed is:

1. A sequential series of individual ankle foot orthotic (AFO) devices that are custom fitted for a foot of a patient and that vary incrementally from one to the next in a flexion angle, the series of devices comprising an initial device, a final device, and at least one intermediate device, each individual AFO device of the sequential series comprising:
a posterior strut defining a vertical axis and having a proximal end and a distal end; and
a foot support portion coupled with the posterior strut at or near its distal end, wherein the foot support portion is custom fitted for the foot of the patient, and wherein the foot support portion comprises;
a foot bed, comprising a bottom surface defining a bottom plane of the individual AFO device; and
an ankle cover removably connected to the foot bed,
wherein a vertex of the vertical axis of the posterior strut and the bottom plane of the foot bed defines the flexion angle, and
wherein an overall configuration of each individual AFO device of the sequential series of AFO devices is determined at least in part by a single digital profile of the foot of the patient.

2. The sequential series of AFO devices of claim 1, wherein a difference between the flexion angles of the initial device and the final device, respectively, is between 80° and 110°, and wherein an incremental flexion angle difference between individual sequential neighboring AFO devices within the sequential series is between 1° and 10°.

3. The sequential series of AFO devices of claim 1, wherein a configuration of a distal portion of the posterior strut varies between at least some of the AFO devices in the sequential series, and wherein the varied configuration of the distal portion affects the flexion angles of the AFO devices.

4. The sequential series of AFO devices of claim 1, each individual AFO device further comprising a leg support connected to a proximal portion of the posterior strut.

5. The sequential series of AFO devices of claim 1, wherein the posterior strut of each individual AFO device comprises a thermoplastic composition.

6. The sequential series of AFO devices of claim 5, wherein the thermoplastic composition comprises a thermoplastic fiber composite composition, the fiber comprising continuous fiber.

7. The sequential series of AFO devices of claim 1, wherein the posterior strut of each individual device is drawn from a collection of posterior struts that vary in size and shape.

8. The sequential series of AFO devices of claim 1, wherein the foot support portion of each individual AFO device comprises a material selected from the group consisting of a thermoplastic composition and a thermoset composition.

9. The sequential series of AFO devices of claim 1, wherein at least one of the foot bed portion or the ankle cover portion of each individual AFO device comprises a thermoplastic composition and is formed by a direct molding process against the foot of the patient.

10. The sequential series of AFO devices of claim 1, wherein at least one of the foot bed portion or the ankle cover portion of each individual AFO device is formed by way of a 3D printed mold, the 3D printed mold being derived from the single digital profile of the foot.

11. The sequential series of AFO devices of claim 1, wherein at least one of the foot bed portion or the ankle cover portion of each individual AFO device is formed by way of a 3D printing process, as directed by the single digital profile of the foot.

12. A sequential series of individual ankle foot orthotic (AFO) devices that are custom fitted for a foot of a patient and that vary incrementally from one to the next in a flexion angle, the series of devices comprising an initial device, a final device, and one or more intermediate devices, each individual AFO device of the sequential series comprising:
an integrated posterior support/foot bed portion that defines the flexion angle; and
an ankle cover portion removably coupled with and disposed over the integrated posterior support/foot bed portion,
wherein the posterior support/foot bed portion and the ankle cover portion are both custom fitted for the foot of the patient, and
wherein a single digital profile of the foot of the patient serves as a model for the fabrication of each individual AFO device within the sequential series of the AFO devices.

13. The sequential series of AFO devices of claim 12, wherein a difference between the flexion angles of the initial device and the final device, respectively, flexion angle is between 80° and 110°, and wherein an incremental flexion angle difference between individual sequential neighboring AFO devices within the sequential series is between 1° and 10°.

14. The sequential series of AFO devices of claim 12, wherein the posterior support/foot bed portion of each individual AFO device comprises a thermoplastic composition.

15. The sequential series of AFO devices of claim 14, wherein the thermoplastic composition comprises a thermoplastic fiber composite composition, the fiber comprising continuous fiber.

16. The sequential series of AFO devices of claim 12, wherein at least one of the posterior support/foot bed portion or the ankle cover portion of each individual AFO device comprises a thermoplastic composition and is formed by a direct molding process against the foot of the patient.

17. The sequential series of AFO devices of claim 12, wherein at least one of the posterior support/foot bed portion or the ankle cover portion of each individual AFO device is formed by way of a 3D printed mold, the 3D printed mold being derived from the single digital profile of the foot.

18. The sequential series of AFO devices of claim 12, wherein at least one of the posterior support/foot bed portion or the ankle cover portion of each individual AFO device is formed by way of a 3D printing process, as directed by the single digital profile of the foot.

19. A method of fabricating a sequential series of orthopedic devices custom designed to change a configuration of a body part of a patient from a pretreatment configuration to a treated configuration, the method comprising:
receiving digital data representing the body part of the patient in the pretreatment configuration;
generating, using the digital data, a sequential series of digital 3D body part models, including at least an initial body part model representing the pretreatment configuration of the body part, a final body part model representing the treated configuration of the body part, and at least one intermediate body part model representing the body part in an intermediate configuration between the pretreatment and treated configurations; and
fabricating the sequential series of orthopedic devices from the sequential series of digital 3D body part models.

20. The method of claim 19, wherein the sequential series of orthopedic devices comprises a sequential series of ankle foot orthosis (AFO) devices.

21. The method of claim 20, wherein the initial, final and at least one intermediate body part models vary, relative to one another, in a flexion angle.

22. The method of claim 21, wherein the flexion angle change throughout the sequential series of AFO devices is between 80° and 110°, and wherein an incremental difference between any two adjacent devices within the sequential series is between 1° and 10°.

23. The method of claim 19, wherein receiving the digital data comprises receiving 3D imaging data acquired using an imaging modality selected from the group consisting of CT and MRI.

24. The method of claim 23, wherein receiving the digital data comprises receiving a 3D profile of the body part in the form of an STL file.

25. The method of claim 24, further comprising, after the receiving step, importing the STL file into a CAD application, wherein the generating step is performed using the CAD application.

26. The method of claim 25, further comprising, after the generating step, importing the sequential series of body part models into an STL CAD manipulation application.

27. The method of claim 19, wherein body part comprises an upper limb or a lower limb, and wherein the method further comprises repeating the method steps for a contralateral upper limb or lower limb to provide a second sequential series of orthopedic devices for the contralateral upper limb or lower limb.

28. The method of claim 27, wherein the sequential series and the second sequential series of orthopedic devices comprise ankle foot orthosis (AFO) devices, and wherein the two sequential series are configured for left and right feet of the patient.

29. The method of claim 19, wherein fabricating the sequential series of orthopedic devices comprises at least one of 3D printing or 3D machining.

30. The method of claim 19, wherein fabricating the sequential series of orthopedic devices comprises:
forming a sequential series of positive molds from the sequential series of digital 3D body part models; and
forming the sequential series of orthopedic devices from the sequential series of positive molds.

31. The method of claim 19, wherein fabricating the sequential series of orthopedic devices comprises:
forming a sequential series of negative molds from the sequential series of digital 3D body part models; and
forming the sequential series of orthopedic devices from the sequential series of negative molds.

32. The method of claim 19, wherein fabricating the sequential series of orthopedic devices comprises forming the sequential series of orthopedic devices directly from the sequential series of digital 3D body part models, without using any molds.

33. The method of claim 19, further comprising:
receiving additional digital data representing the body part of the patient after treatment of the body part has commenced; and
repeating the generating and fabricating steps to make at least one additional orthopedic device to further treat the body part.

34. The method of claim 19, further comprising receiving a treatment plan from a physician, wherein the treatment plan comprises at least one parameter defining the treated configuration of the body part.

35. The method of claim 34, further comprising receiving a follow-up treatment plan from the physician during treatment of the patient, wherein the follow-up treatment plan includes at least one instruction for altering a planned sequential series of orthopedic devices.

36. The method of claim 35, wherein the follow-up treatment plan comprises receiving a second set of digital data representing the body part of the patient prior to concluding the treatment as originally planned.

37. The method of claim 19, wherein the at least one intermediate body part model comprises multiple, sequential, intermediate body part models.
